(19) United States
(12) Patent Application Publication
(10) Pub. No.: US 2008/0020041 A1
(43) Pub. Date: Jan. 24, 2008

(54) Enteric Coated Compositions that Release Active Ingredient(s) in Gastric Fluid and Intestinal Fluid

(76) Inventor: James W. Ayres, Corvallis, OR (US)

Correspondence Address: KLARQUIST SPARKMAN, LLP, 121 SW SALMON STREET, SUITE 1600, PORTLAND, OR, 97204, US

(21) Appl. No.: 11/665,729

(22) PCT Filed: Oct. 3, 2005
(86) PCT No.: PCT/US05/35787

§ 371 Date: Apr. 18, 2007

Related U.S. Application Data
(60) Provisional application No. 60/620,482, filed on Oct. 19, 2004.

(51) Int. Cl. A61K 9/24; A61K 33/24; A61K 33/08; A61K 48/00

(52) U.S. Cl. 424/472; 514/44; 424/692; 424/653; 514/573; 514/252.17; 514/338; 514/649

(57) ABSTRACT

Embodiments of a pharmaceutical formulation comprising an enteric material are disclosed. The embodiments release at least a portion of an active ingredient upon contacting gastric fluid. The remaining portion of the formulation releases active ingredient upon contacting intestinal fluid. Certain embodiments of the pharmaceutical composition comprise at least one active ingredient in a core and a leaky enteric coating, such as an enteric coating comprising a gastric fluid channeling agent. Other embodiments of the pharmaceutical composition comprise at least one active ingredient substantially homogeneously admixed with at least one enteric material, such as an enteric material comprising a gastric fluid channeling agent. Disclosed embodiments of the pharmaceutical composition may comprise a single active ingredient, or may comprise plural active ingredients. Generally, but not necessarily, the active ingredient has a window of absorption. The present disclosure also describes a method for treating a subject having a condition treatable by an active ingredient. The method comprises providing one or more embodiments of the pharmaceutical composition disclosed herein comprising an active ingredient suitable for treating the condition. The pharmaceutical composition is administered to the subject. A method for making embodiments of the disclosed composition also is described. The method comprises providing a core comprising an active ingredient. An enteric material is applied to at least a portion of the core, and generally on or about a substantial portion of the core, to form a coat. The composition is then made leaky.

### CROSS REFERENCE TO RELATED APPLICATION

This application claims the benefit of the earlier filing date of U.S. Provisional Application No. 6/620,482, filed on Oct. 19, 2004. The entire disclosure of provisional application No. 60/620,482 is considered to be part of the disclosure of the accompanying application and is incorporated herein by reference.

### FIELD

The present disclosure concerns pharmaceutical compositions, methods for preparing such compositions, and methods for their use, particularly orally administered dosage forms having active agents with site specific absorption and enteric coats that release at least a portion of the active agents in acidic gastric fluids.

### BACKGROUND

Enteric coating of dosage forms that contain drugs is well known in the pharmaceutical sciences literature. Enteric coatings are coatings designed to prevent release of the enteric-coated drug in gastric fluid of the stomach and prevent exposure of the drug to the acidity of the gastric contents while the enteric coated drug composition is in the stomach. After passing from the stomach into the intestine, the enteric coating dissolves and releases the drug into intestinal fluids.

The Food and Drug Administration (FDA) defines drug dosage forms that are enteric coated as “delayed-release” dosage forms. Delayed-release (enteric coated) dosage forms are differentiated from controlled-release or sustained-release dosage forms, which are intended to provide drug input over an extended period of time, thereby reducing administration frequency. FDA guidelines for enteric-coated dosage forms state: “In vitro dissolution tests for these products should document that they are stable under acidic conditions and that they release the drug only in a neutral medium (e.g., pH 6.8).”

A. Drugs with an Absorption Window

Site specific absorption for orally administered therapeutics refers to therapeutic agents that are absorbed only from or better from one region or area of the intestinal tract relative to other areas or regions of the intestinal tract. Most drugs are not well absorbed from the stomach and are well absorbed from the small intestine. Many drugs also are well absorbed from the colon. Drugs that are absorbed throughout the intestine, including the colon, often are good candidates for sustained drug release formulations, especially if such drugs have a relatively short biological half-life. Sustained-release, drug formulation product literature provides numerous examples of such formulations.

Some drugs that undergo site specific absorption are only absorbed or are best absorbed in the small intestine. Such drugs may pass the absorption site without being available such as, for example, when trapped inside a dosage formulation or the drug is slowly soluble and has not yet had time to dissolve. Any drug that has not been absorbed at the adsorption site will not be absorbed, or is absorbed so slowly or so poorly that it is not therapeutically effectively available to the body. In these cases the bioavailability of the drug is incomplete. Some drugs undergo site specific drug absorption because their absorption involves transporter systems that are concentrated in certain regions of the intestinal tract. In this case if the drug is delivered into the absorption site area faster than drug can be absorbed, then the transporter system may become saturated. Any additional drug that is present is not carried across the membrane by the transporter system but travels on unabsorbed. The result is incomplete bioavailability. Such site specific absorption drugs, regardless of the mechanism associated with site specific absorption, are said to have an absorption window. The absorption window may be, and commonly is, in the jejunum, the duodenum, or a combination thereof.

B. Discussion of Patents and Publications

U.S. Pat. No. 6,399,086 teaches that β-lactam antibiotics have a specific absorption site in the small intestine. The '086 patent also teaches that there is a need for a dosage form that provides about 50% of the drug within 3-4 hours of administration, and releases the remainder of the drug at a controlled rate. Such dosage form may comprise a β-lactamase inhibitor. The “Background” section of the '086 patent teaches that enteric coating controlled release amoxicillin trihydrate suppresses drug release at gastric pH, but that this result is not useful. The '086 patent states that:

Hilton and Deasy [J. Pharm. Sci. 82(7):737-743 (1993)] described a controlled-release tablet of amoxicillin trihydrate based on the enteric polymer hydroxy-propylmethyl cellulose acetate succinate. This polymer suppressed the release of the drug in the presence of gastric pH but could enhance its release in the small intestine. Therefore, such a formulation cannot give the desired burst effect discussed below. Single dose studies with a panel of fasting subjects showed that the tablets had a relative bioavailability of only 64.4%, probably because the poorer absorption of amoxicillin from the distal jejunum and ileum than from the duodenum and proximal jejunum. Other pharmacokinetic parameters confirmed a lack of therapeutic advantage of these factors over an equivalent dose of conventional capsule.

The '086 patent provides additional information about drugs that have an absorption window and gives examples of some drugs that are useful in the instant invention disclosed herein. The '086 patent is incorporated herein by reference in its entirety. The '086 patent further states that:
The term “drugs which have an absorption window” as currently used in the art refers to drugs which are absorbed at specific sites in the gastrointestinal tract, for example drugs which are absorbed by carrier-mediated mechanisms. Such mode of absorption is suggested for compounds like dipeptides [Matthews, D. M., Biochem. Soc. Trans. 11:808-810 (1983)], riboflavin [Levy, J. & W. Jusko, J., J. Pharm. Sci. 55:285-289 (1966)], folic acid [Hepner, G. W. et al., Lancet 2:302-306 (1968)] and ascorbic acid [Mayersohn M., Eur. J. Pharmacol. 19:140-142 (1972)]. Penicillin may be regarded as a dipeptide derived from cysteine and valine [Doyle, F. P. & Nayler, J. H. C., Advances in Drug Research 1:8-13, (1964); Harper, N. J. & Simmonds, A. B. Eds. Academic Press] and is thus absorbed by a special transport mechanism common to the absorption mechanism of dipeptides, for which a suitable transport system has been demonstrated in man [Matthews, D. M., ibid.; Silk, D. B. A. et al., Ann. Nutr. Metab. 26:337-352 (1982)].
Another mechanism for drugs which are absorbed at a specific absorption site is associated with drugs which are solubilized at a specific locus in the gastrointestinal tract, for examples fats. A specific illustrative example for this mechanism is tocopherol which is solubilized by bile acid micelles [Guyton, A. C., Textbook of Medical Physiology, W.B. Saunders Company, (1986)]. Further examples of drugs which are absorbed by carriers are salts [Guyton, A. C., (1986) ibid.] AZT, 5-FU, .alpha.-methyl-Dopa and L-Dopa, riboflavin [Gibaldi, M., Biopharmaceutics and Clinical Pharmacokinetics, 3rd Edition, (1984); Evans, W. E. et al., Applied Pharmacokinetics 19:1-14 (1992); Rowland, M. & Tozer, T. N., Clinical Pharmacokinetics, concepts and applications, pp 23-24, (1988), Lea & Febiger, Philadelphia]. According to the preferred embodiments of the present invention, the β-lactam antibiotic drug capable of providing the desired burst effect, is cephalosporin and/or a penicillin. Examples of cephalosporins which may be used with the delivery system of the invention are cefadroxil, cefalexin, cefaclor, cefprozil, cefuroxime, cefoxitin, cefpodoxime, cefixime, pharmaceutically acceptable salts thereof or pharmaceutically acceptable derivatives thereof. Examples of penicillins which may be used in the delivery system of the invention are penicillin G, penicillin V, amoxicillin, ampicillin, nafcillin, oxacillin, cloxacillin, dicloxacillin, or pharmaceutically acceptable salts thereof. Examples for pharmaceutically acceptable cephalosporin derivatives, which may be used in the delivery system of the invention, are cefpodoxime proxetil and cefuroxime axetil.
In a particularly preferred embodiment of the present invention the drug delivery system of the invention contains as the β-lactam agent amoxicillin trihydrate or pharmaceutically acceptable salts thereof. A mixture of the active antibiotic agent with a pharmaceutically acceptable salt thereof can also be used as the active pharmaceutical agent in the delivery system of the invention. Thus, for example, it may be advantageous to use a mixture of amoxicillin with amoxicillin sodium salt in order to provide the desired burst effect.
It may be advantageous to add to the drug delivery systems of the invention a lactamase inhibitor. Suitable β-lactamase inhibitors are clavulonic acid or sulbactam. β-lactamase inhibitors themselves have poor antibacterial activity. However, when given in combination with penicillins, to treat infections involving β-lactamase producing bacteria, they enhance the antibiotic effect [Kalant, H. & Roschlau, W. A. E., Principles of Medical Pharmacology, 5th Ed., pp 549 (1989) B. C. Decker Inc.].
The drug delivery system of the invention may further optionally contain additional pharmaceutical agents having a specific absorption site in the small intestine, for example, vitamins such as riboflavin, folic acid, ascorbic acid, thiamin or tocopherol or mixtures thereof, anti-viral agents such as AZT, antitumor agents, therapeutic metal inorganic salts such as iron salt, lithium salt or potassium salt, antihypertensive agents such as .alpha.-methyl Dopa and antiparkinsonian agents such as L-Dopa. The drug delivery system of the invention may also fiber contain a mixture of such agents, e.g. a mixture of vitamin/s with other therapeutic agents, for example metals. A specific example may be a mixture of iron and folic acid.”

International patent application No. PCT/US01/20134 provides information concerning oral sustained release formulations (SR) that are designed to provide slow drug release over time periods of 4 or 6 or more hours, such as well-known, ethyl-cellulose-coated bead formulations, osmotic pump tablets, hydrophilic matrix compressed tablets and the like, and further teaches that such formulations are not suitable for drugs that have an absorption window. Such sustained release dosage formulations are well known to be transported, after leaving the stomach, through the small intestine and past known absorption window areas in about 3-5 hours. International patent application No. PCT/US01/20134 states that:
That is, after transiting the stomach, there is an approximately 3-5 hour window of bioavailability before the dosage form reaches the colon. Sustained or delayed release vehicles that are not retained in the stomach before and during release of the drug may release a significant portion of the drug after the window of bioavailability has passed.
   Thus, any drug with an absorption window trapped in the dosage formulation is likely to not be bioavailable. Total transit times from stomach-to-colon after swallowing a drug dosage formulation are variable, depending mostly on whether or not there is food in the stomach, and stomach emptying time. Transit times in the small intestine are relatively uniform. Even if a patient might eat frequently enough to interrupt the body's “house keeper wave” that empties the stomach, drugs with an absorption window still are not suitable for known sustained release formulations because it is too likely that the drug formulation will be trapped in the stomach. Alternatively, the drug formulation will leave the stomach quickly. These release patterns result in highly variable bioavailability from dose-to-dose or day-to-day, and increase the occurrence of drug toxicity or clinical failure.

Thus, a need exists for a composition that will provide good bioavailability of drugs that have an absorption window. There is an additional need for compositions that increase the duration of action or decrease the frequency of dosing of absorption-window drugs even if they do not increase or maintain bioavailability for drugs with a window of absorption. Such compositions have not been known but are disclosed herein. And, it is additionally surprising that the new compositions are useful for drugs with an absorption window not only if the absorption is known to be limited by transporter systems saturation or because the drug is slowly soluble.

WO 02/00213 A1 PCT/US01/2013 describes a rapidly expanding composition for gastric retention that can provide controlled release of therapeutic agents in the stomach. The primary feature of such an expanding dosage form is that it is retained in the stomach because of its large size. The primary value of such an expanding dosage form is delivery of drugs that are most readily absorbed by the jejunum and duodenum, i.e., drugs with an absorption window. A disadvantage of such dosage forms is that they are “single unit”, i.e., a single tablet or a single capsule. It is well known that single-unit dosage forms often provide a “partial or none” effect. Single-unit tablet or capsule gastric retention devices may provide the desired effect when administered with food but have been shown to be removed from the stomach by the Intermittent Migrating Myoelectric Complex, commonly known as the “housekeeper wave”. Multiple-unit drug dosage forms such as multiple pellets or beads inside a capsule provide a distinct advantage over single-unit dosage forms because the average effect of the beads is to release drug even if a single bead fails to be an effective delivery unit.

C. Enteric Coated Formulations

Enteric formulations and enteric coated formulations are well known in the pharmaceutical sciences and medical practice. Enteric coatings are intended to protect the therapeutic agent from destruction or degradation by the acid contents of the stomach or to prevent the therapeutic from irritating the stomach, and delays release of the therapeutic until such time as the enteric-coated formulation reaches the intestine. Enteric formulations then allow the therapeutic to be released into the less acidic fluids of the intestinal tract. See, for example, Remington's Pharmaceutical Sciences, 18^{th} Ed., page 1634 (Mack Publishing, 1990), which states: “Enteric-Coated Tablets (ECT)—These are compressed tablets coated with substances that resist solution in gastric fluid but disintegrate in the intestine.” Enteric coating is not only applied to tablets, but also is commonly applied to beads to prevent exposing therapeutic to gastric acid as is shown in U.S. Pat. No. 4,786,505.

The FDA publishes industry guidelines. For example, Guidance for Industry, Bioavailability and Bioequivalence Studies for Orally Administered Drug Products-General Considerations

(http://63.75.126.224/Google/fda_search .p1?client=fdagov&site=fdagov&searchselector=&g=enteric%2C+delayed&sa=Search&restrict=cder_guidance) states that:

As defined in the U.S. Pharmacopeia (USP), delayed-release drug products are dosage forms that release the drugs at a time later than immediately after administration (i.e., these drug products exhibit a lag time in quantifiable plasma concentrations). Typically, coatings (e.g., enteric coatings) are intended to delay the release of medication until the dosage form has passed through the acidic medium of the stomach. In vitro dissolution tests for these products should document that they are stable under acidic conditions and that they release the drug only in a neutral medium (e.g., pH 6.8).

The literature clearly teaches that a sufficient amount of enteric coating must be utilized to produce an acceptable enteric-coated drug product that does not release drug in gastric fluid. An insufficient amount of enteric coating material is taught to be unacceptable. A leading manufacturer of enteric coating polymers, Rohm Pharma Polymers, recommends a 30- to 50-micron thickness coating in order to obtain adequate enteric coating protection (Eudragit® technical sheets, Rohm Tech Inc., MA).

Also, U.S. Pat. No. 6,605,300 and WO 2000023055 A1 teach that:
Typical enteric coating levels did not meet the above requirements for the desired dosage profile of amphetamine salts. Using the typical amount of enteric coating (10-20.mu.) resulted in undesired premature leakage of the drug from the delivery system into the upper gastrointestinal tract and thus no drug delivery at the desired location in the gastrointestinal tract after the appropriate lag time. The unacceptable premature drug release from the delivery system in gastric fluid and no drug delivery to the desired location in the gastrointestinal tract after an appropriate delay time teaches such coatings are not acceptable.

“EUDRAGIT® L 30D-55 (Rohm Pharma, Germany) coating dispersion” was used in the first example. An enteric layer 20-microns thick on drug-loaded beads resulted in unacceptable premature drug release from the delivery system in gastric fluid and no drug delivery to the desired location in the gastrointestinal tract after an appropriate delay time. Thus this coating did not meet the requirements of an enteric coating. Applicants then report that a thicker application of an enteric coating having a thickness of greater than 25 microns was required to provide an effective enteric coat.

For pharmaceutical formulations known prior to the present invention, enteric coatings must protect drug and drug must not be released in gastric fluid for particular periods of time to be considered effective. For example, Jorg Brietkreutz concludes that “[t]he criteria of the pharmacopeias are usually set to 2 or 3 hours of gastric juice resistance. Sometimes 1 hour is accepted in exceptional cases.” See, Jorg Brietkreutz, Leakage of enteric (Eudragit L)-coated dosage forms in simulated gastricjuice in the presence of poly(ethylene glycol), Journal of Controlled Release, 67 (2000) 79-88). Brietkreutz also states that “recently, 40-55 mm was reported to be the minimum coating thickness for Eudragit L”. And, commercially available enteric coated products containing omeprazole are reported unstable when both PEG and enteric coated products are in gastric fluid because “In the case of the tablet with micropellets, omeprazole release starts at about 100 min, whereas the capsule formulation releases the drug after 150 minutes.” The amount of drug released in gastric fluid for the authors to conclude the products are unstable is less than 10%. This teaching that less than 10% drug release in gastric fluid indicates an unstable or unsuitable enteric coat is consistent with FDA guidelines requiring that enteric coats only release drug in neutral medium and not acidic medium. Further, United States Pharmacopea indicates that the maximum amount of drug release allowable from an enteric-coated product is 10% for dissolution testing in simulated gastric fluid for 2 hours. U.S. Pharmacopeia, 23, U.S. Pharmacopeial Convention: Rockville, Md., 1994, pp. 1795-1796.

Riboflavin, a drug with an absorption window, has been formulated as enteric-coated pellets (H X Guo, J Heinamaki, and J Yliruusi, Diffusion of a Freely Water-Soluble Drug in Aqueous Enter-Coated Pellets, AAPS Pharm Sci Tech, 2003:3(2) article 16 (http://www.aapspharmscitech.org). The effects of pellet filler and enteric-coating thickness on drug release in gastric fluid were studied. When the core pellet contained waxy cornstarch, a 20% weight gain of traditional enteric coating was reported to have “failed the test” by releasing about 20% drug in gastric fluid in 1 hour, but 30% enteric coating did prevent drug release. The authors state that “[n]either the 20% nor the 30% enteric-coated lactose pellets gave acidic resistance”, releasing about 45% and 55% drug in gastric fluid in 1 hour. The authors go to great lengths to study the reasons and mechanisms involved in how the pellet core composition causes the enteric coating to fail and conclude that diffusion of a water-soluble drug and excipient into an enteric coating can result in “coating failure and, subsequently, premature dissolution of enteric-coated pellets in an acidic environment”. Clearly, persons of ordinary skill in the art regard “coating failure” and “premature dissolution” as results to be avoided.

Beckert et al. describe sucrose pellets having a coating of bisacodyl admixed with Eudragit L 30 D-55. This formulation may further include a coating of methyl methacrylate/methacrylic acid polymers. Beckert et al., Compression of Enteric-Coated Pellets to Disintegrating Tablets, “International Journal of Pharmaceutics 143, pp. 13-23 (1996). With reference to Eudagrit L 30 D-55, the authors conclude that films made from such materials “are so brittle that even the double amount of coating does not reduce the damage with the coatings.” Id. at 21. Beckert et al. apparently desired compounds that release less than 10% bisacodyl in gastric fluid to comply with USP 23, but which release “sufficient bisacodyl between pH 6.8 and 7.5,” i.e. at intestinal pH levels. Beckert et al. state that:
The amount of bisacodyl liberated is reduced with thicker films if coatings containing a mixture of Eudagrit L and Eudgrit NE are applied. The liberation of bisacodyl in 0.1 M HCl from this film is approximately 4% w/w of the total bisacodyl content. As these coatings do not dissolve and release sufficient bisacodyl between pH 6.8 and 7.5, two new polymers (Table 4) showing high elasticity combined with sufficient dissolution in the pH range 6.8-7.0 (FIG. 7) have been developed by Lehmann and Sufke. Emphasis added.
   Id. Thus, Beckert et al. concluded that sucrose beads coated with Eudagrit L admixed with bisacodyl required too thick a coating to provide a useful formulation. The authors therefore further coated the formulation with methyl methacrylate/methacrylic acid polymers. With reference to these formulations, Beckert et al. conclude that:
Pellets coated with 25% w/w of one of the new polymers liberate only 4-5% w/w bisacodyl in acidic media after tableting. The liberation of bisacodyl in phosphate buffer within 45 min at pH 6.8 is 100% for polymer 1 and 40% for polymer 2 and rises to 100% at pH 7.2. Thus, tablets comprising enteric-coated bisacodyl pellets are available which comply with all recommendations of USP 23.
   Id.

Further, the authors conclude that:
The remaining deformation of pellets needs to be neutralized by elastic coatings which can follow the deformation without rupturing. The most important parameters are the type and the applied thickness of the film forming polymer. Disintegrating tablets can be obtained from enteric-coated pellets which do not liberate more than 10% bisacodyl after 2 h in 0.1 m HCl, thus complying with USP 23.
   Id. at 22. Thus, these authors conclude that rupturing coatings so that gastric release of therapeutic, particularly release in excess of the 10% level recited in USP 23, is an undesirable result. Beckert et al. also refer to the work of other authors concerning gastric release of acetylsalicylic acid and indometacin coated with Eudragit L and sulfametoxazole coated with cellulose acetate phthalate. Acetylsalicylic acid and indometacin pellets have been made that release less than 10% w/w of the active ingredient within 2 hours in 0.1 M HCl. These tablets conformed to the requirement of USP 23 for enteric coated preparations. See, Lehmann, et al., Acta Pharm. Technol., 36, 7S (1990); Lehmann et al., Schnellzerfallende Tabletten mit Gesteurter Wirkstoffabgabe, Pharm. Ind., 55, 940-947 (1993). With reference to sulfametoxozole Beckert et al. state that:
Disintegrating tablets from sulfametoxazole pellets coated with cellulose acetate phthalate were described by Takenaka et al. [Preparation of Enteric-Coated Microcapsules for Tableting by Spray-drying Technique and in vitro Simulation of Drug Release from the Tablet in GI-tract, J. Pharm. Sci 9, 1388-1392 (1980)], but liberated more than 10% of the drug within 2 hours in artificial gastric fluid and thus did not conform to the requirement of USP 23.
   Id, at 14.

Thus, the literature teaches that an insufficient amount, or an enteric coating layer that is too “thin” produces an unacceptable, defective enteric coat that allows contact of acidic gastric fluids with therapeutic agents inside the enteric coating and/or allows release of the enteric-coated therapeutic agents into acidic fluid. It also is generally well known in the field that coating drug dosage forms to sustain or delay drug release may reduce the amount of drug absorbed into the body.

U.S. Patent Publication No. 20030021845 A1 discloses a very complex, multilayered, single-unit gastroretentive divice that must be folded prior to administration. The device has multiple layers of polymer sheets, generally glued together by solvent softening. The device is too large to swallow without folding and too large to pass through the pyloric sphincter until delaminated, dissolved, or disintegrated. In the stomach, the device unfolds and slowly degrades or dissolves such that the device is retained in the stomach longer than a conventional dosage form, for a minimum of 3 hours and preferably about 8-12 hours. In some cases a polymer combination involved may be a “shielding layer” optionally covering part or all of the face of other polymer sheets of the device. According to U.S. patent publication No. 20030021845 the shielding layer polymer is selected from “(a) a hydrophilic polymer which is not instantly soluble in gastric fluids; (b) an enteric polymer substantially insoluble at pH less than 5.5; (c) a hydrophobic polymer; and (d) any mixture of at least two polymers as defined in any of (a), (b), or (c).” U.S. Patent Publication No. 20030021845 also states that:
There are several advantages in including an enteric polymer in the matrix or the shielding layer, as enteric polymers have improved mechanical properties (e.g. Young's modulus and yield strength). The addition of an enteric polymer to the shielding layer prevented rapid rupture of the shielding layer in vitro. A further advantage of using an enteric polymer is to ensure the complete dissolution and/or disintegration of the components of the device, e.g. the matrix, the shielding layer or the membrane, in the intestine, had it not already occurred in the stomach. A preferred enteric polymer incorporated into the shielding layer may be methylmethacrylate-methacrylic acid copolymer, at a ratio of 2:1 ester to free carboxylic groups.
   The device described by U.S. Patent Publication No. 20030021845 A1 is intended to be retained in the stomach and to release drug into gastric fluids of the stomach. Thus, the shielding polymer layer must necessarily allow release of drug into gastric fluid even though it comprises an enteric polymer as one of the multilaminated films but such compositions are not known for multiparticulates, such as beads or granules, or for tablets or capsules. The compositions disclosed by U.S. Patent Publication No. 20030021845 A1 differ substantially from the new compositions disclosed herein. For example, the shielding layer of U.S. Patent Publication No. 20030021845 A1 were prepared by casting the polymers in a mixture of 50% ethyl alcohol and 50% NaOH followed by evaporative drying. Dried films were than affixed to other dried films to produce multilaminate sheets using ethyl alcohol to partially “melt” the films together. Polymer films formed by casting as taught could be glued to one or two faces of a tablet and folded like wings to promote gastric retention. But it is not practical or even possible to uniformly coat the entire surface of tablets or particulates, such as beads and granules, using the method taught by the published application.

Tablets, beads, granules, capsules, and active ingredients would dissolve and/or degrade if mixed into such a solution for casting. There is no known useful commercial way to affix such films to uniformly coat the substantially round or irregularly shaped tablets, beads, granules, or capsules.

Gastroretentive devices can be sustained-release dosage forms because they reduce the required frequency of dosing for some drugs. Another way to reduce drug dosing frequency is to formulate what has been called a “pulse” drug delivery system using a mixture of a fixed ratio of immediate release and enteric-coated drug. In this system, 50% of the dose is released immediately in gastric fluid (the first pulse) and 50% is enteric coated and then released after transfer from the stomach into the intestine (the second pulse). U.S. Pat. No. 6,322,819 teaches a “pulsed dose delivery” is important for amphetamines. The '819 patent teaches that typical enteric coating levels on amphetamine-loaded pellets resulted in undesired premature leakage of drug in the upper intestinal tract, and thus did not provide drug delivery at the desired location in the gastrointestinal tract after the appropriate lag time. An enteric coating thickness of at least 25 μm was required to prevent premature drug leakage. Then, essentially all the enteric-coated drug was released within 1 hour after transfer into intestinal fluid. This combination of 50% immediate release of drug and 50% enteric-coated drug that did not release in gastric fluid resulted in a pharmacokinetic drug pattern that allows a reduction in dosing frequency. The '819 patent states that “it will be appreciated that the multiple dosage form of the present invention can deliver rapid and complete dosages of pharmaceutically active amphetamine salts to achieve the desired levels of the drug in a recipient over the course of about 8 hours with a single oral administration.” Similar results have been obtained for cyclosporine, a drug with a window of absorption, when formulated as 50% dried microemulsion and 50% dried enteric coated micoemulsion. See, Chong-Kook Kim, Hee-Jong Shin, Su-Geun Yang, Jae-Hyun Kim and Yu-Kyoung Oh, Once-a-Da Oral Dosing Regimin of Cyclosporin A: Combined Therapy of Cyclosporin A Premicoemulsion Concentrates and Enteric Coated Solid-State Premicoemulsion Concentrates, Pharmaceutical Research, Vol. 18, No. 4, 2001 (454-459)). Enteric coated compositions deemed acceptable by the authors prevented all drug release during 2 hours of exposure to gastric fluid.

D. Gastrointestinal Tract Transit Times

GI transit time of drug pellets has been extensively studied. Food was shown to have a profound effect on gastric emptying rate of drug pellets. Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340.; Davis, S. S.; Khosla, R.; Wilson, C. G.; Washington, N. Gastrointestinal transit of a controlled-release pellet formulation of tiaprofenic acid and the effect of food. Int. J. Pharm. 1987. 35, 253-258.; Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.)

In the fed condition, the gastric emptying rate of pellets appears to be zero order over 5 to 8 hours. (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.; Fischer, W.; Boertz, A.; Davis, S. S.; Khosla, R.; Cawello, W.; Sandrock, K.; Cordes, G. Investigation of the gastrointestinal transit and in vivo drug release of isosorbide-5-nitrate pellets. Pharm. Res. 1987. 4 (6), 480-485.; Bechgaard, H.; Christensen, F. N.; Davis, S. S.; Hardy, J. G.; Taylor, M. J.; Whalley, D. R.; Wilson, C. G. Gastrointestinal transit of pellet systems in ileostomy subjects and the effect of density. J. Pharm. Pharmacol. 1985. 37, 718-721.)

These findings are consistent with another study that found emptying of solids is approximately a zero-order function. (Collins, P. J.; Horowitz, M.; Cook, D. J.; Harding, P. E.; Shearman, D. J. C. Gastric emptying in normal subjects—a reproducible technique using a single scintillation camera and computer system. Gut 1983. 24, 1117-1125.)

Meal size influences the half-time by which pellets are emptied gastrically. The mean half-time was 78 minutes after a light meal (1,500 kJ or 358.5 kcal) compared to 170 minutes for a heavy meal (3,600 kJ or 860.4 kcal). (Davis, S. S.; Khosla, R.; Wilson, C. G.; Washington, N. Gastrointestinal transit of a controlled-release pellet formulation of tiaprofenic acid and the effect of food. Int. J. Pharm. 1987. 35, 253-258.) In the fasted condition, fifty percent of ingested pellets were emptied from the stomach within an hour with a range of less than 0.3 to 0.9 hour (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.) depending upon the time of administration relative to an occurrence of phase 3 of the migrating myoelectric complex (MMC). (Mayer, E. A. The physiology of gastric storage and emptying. In Physiology of the gastrointestinal tract; Johnson, L. R., Ed.; Raven Press: New York, 1994; 929-976.)

It is known that emptying of non-nutrient-containing liquid appears to be first order and volume-sensitive mechanisms play the major role in the regulation of gastric emptying. (Mayer, E. A. The physiology of gastric storage and emptying. In Physiology of the gastrointestinal tract; Johnson, L. R., Ed.; Raven Press: New York, 1994; 929-976.)

Patterns of gastric emptying of pellets taken before a meal were shown to be approximately exponential, i.e., typical of gastric emptying of liquid. (O'Reilly, S.; Wilson, C. G.; Hardy, J. G. The influence of food on the gastric emptying of multiparticulate dosage forms. Int. J. Pharm. 1987. 34, 213-216.)

Lag time of gastric emptying for solid food also differs from that for liquid. The initial lag phase has been observed for gastric emptying of solid food and the average values range from 21 to 60 minutes. (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.; Collins, P. J.; Horowitz, M.; Cook, D. J.; Harding, P. E.; Shearman, D. J. C. Gastric emptying in normal subjects—a reproducible technique using a single scintillation camera and computer system. Gut 1983. 24, 1117-1125.; Mayer, E. A.; Thomson, J. B.; Jehn, D.; Reedy, T.; Elashoff, J.; Deveny, C.; Meyer, J. H. Gastric emptying and seiving of solid food and pancreatic and biliary secretions after solid meals in patients with nonresective ulcer surgery. Gastroenterology 1984. 87, 1264-1271.)

This lag time reflects primarily the time required to reduce the solid food to smaller sizes. (Weiner, K.; Graham, L. S.; Reedy, T.; Elashoff, J.; Meyer, J. H. Simultaneous gastric emptying of two solid foods. Gastroenterology 1981. 81, 257-266.) After a capsule containing drug pellets was administered in the fed condition, seven of eight subjects showed no gastric emptying of the pellets during the first hour. (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75. ) This observed pellet emptying delay suggests that, following capsule disintegration, the pellets became dispersed within the stomach and were mixed with food content before being emptied along with the meal. (O'Reilly, S.; Wilson, C. G.; Hardy, J. G. The influence of food on the gastric emptying of multiparticulate dosage forms. Int. J. Pharm. 1987. 34, 213-216.) Lag time of gastric emptying in these subjects causes lag time of absorption for drug in enteric-coated pellets. (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.) Unlike solid food, liquid emptying has minimal observable lag time. (Collins, P. J.; Horowitz, M.; Cook, D. J.; Harding, P. E.; Shearman, D. J. C. Gastric emptying in normal subjects—a reproducible technique using a single scintillation camera and computer system. Gut 1983. 24, 1117-1125.) Thus, the drug onset action rate is faster for drug dissolved in liquids in the stomach than when drug is trapped inside enteric coated pellets or tablets.

While the presence of food increases the mean gastric emptying time of pellets, the small intestinal transit time is unaffected. (Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340.) The mean small intestinal transit time is about 3 to 4 hours (Shargel, L.; Yu, A. Applied biopharmaceutics and pharmacokinetics. 4th Ed, ed.; Mehalik, C. L.; McGraw-Hill Companies, Inc.: New York, 1999.; Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340.; Davis, S.S.; Khosla, R.; Wilson, C. G.; Washington, N. Gastrointestinal transit of a controlled-release pellet formulation of tiaprofenic acid and the effect of food. Int. J. Pharm. 1987. 35, 253-258.; Fischer, W.; Boertz, A.; Davis, S. S.; Khosla, R.; Cawello, W.; Sandrock, K.; Cordes, G. Investigation of the gastrointestinal transit and in vivo drug release of isosorbide-5-nitrate pellets. Pharm. Res. 1987. 4 (6), 480-485.) and independent of the feeding state. (Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340.; Davis, S. S.; Khosla, R.; Wilson, C. G.; Washington, N. Gastrointestinal transit of a controlled-release pellet formulation of tiaprofenic acid and the effect of food. Int. J. Pharm. 1987. 35, 253-258. Multiple-unit pellets and non-disintegrating single-unit tablets have similar small intestinal transit time. (Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340). Depending on the feeding state, the mean time for the arrival at caecum of pellets ranges from 4 to 8 hours. (Shargel, L.; Yu, A. Applied biopharmaceutics and pharmacokinetics. 4th Ed, ed.; Mehalik, C. L.; McGraw-Hill Companies, Inc.: New York, 1999.; Davis, S. S.; Hardy, J. G.; Taylor, M. J.; Whalley, D. R.; Wilson, C. G. The effect of food on the gastrointestinal transit of pellets and an osmotic device (Osmet). Int. J. Pharm. 1984. 21, 331-340.; Davis, S. S.; Khosla, R.; Wilson, C. G.; Washington, N. Gastrointestinal transit of a controlled-release pellet formulation of tiaprofenic acid and the effect of food. Int. J. Pharm. 1987. 35, 253-258.; Fischer, W.; Boertz, A.; Davis, S. S.; Khosla, R.; Cawello, W.; Sandrock, K.; Cordes, G. Investigation of the gastrointestinal transit and in vivo drug release of isosorbide-5-nitrate pellets. Pharm. Res. 1987. 4 (6), 480485.)

Thus, drug release and effect onset time from enteric-coated compositions is highly variable. Drug release and effect onset time depend on a number of factors, including whether: (1) the composition is a relatively large unit dosage composition, such as a single enteric-coated capsule or tablet; (2) whether the composition is a multiplicity of enteric particulates, such as enteric-coated beads or granules; (3) there is food in the stomach at the same time the composition is in the stomach, and how much time elapses before the housekeeper wave transports the composition into the intestine. Then, there is still some additional lag time until the enteric composition actually starts releasing drug. In some cases, an enteric composition may not release drug for 12 or more hours following administration. The new compositions disclosed herein decrease variability in drug release and onset time by avoiding or minimizing the effect of food to delay drug release by trapping the composition in the stomach. These new compositions still may be trapped in the stomach, but drug release occurs at least partially in the stomach and is not entirely delayed until the composition reaches the intestine.

U.S. Pat. No. 5,232,704 teaches that prostoglandins are principally absorbed from the stomach and hence there is a need to prolong the time such drugs are delivered in the stomach fluid. Moreover, in vivo studies with buoyant, single-unit dosage forms indicate that a mean gastric residence time ranging between 3 and 4 hours can be obtained with fed subjects (light breakfast).

### SUMMARY

Disclosed embodiments of the present invention are directed primarily to drugs that are best absorbed from the upper intestine. But novel enteric compositions that release drug in the stomach also are ideal for drug delivery, such as mistoprostol and other prostaglandins that have a direct action on cells in the stomach or are best absorbed from the stomach. And desirable combinations of drugs, such as, for example, those taught in U.S. Pat. No. 5,232,704, incorporated herein by reference in its entirety, also are advantageously prepared with the novel enteric compositions that release drug in the stomach. In one preferred embodiment, a drug that has a direct action on cells in the stomach or is best absorbed from the stomach, or any other therapeutic agent that is preferably released in the stomach, is combined with other active agents, if desired, and formulated as a single-unit, enteric-coated dosage form, such as a tablet or a capsule that releases drug in gastric fluid. This dosage form preferably is administered before, during, or after a meal such that food is present in the stomach at the same time as the dosage form. The combination of food and a traditional, enteric-coated, single-unit dosage form, especially when the dosage form is a relatively large size, such as commonly used intermediate or large tablets and capsules, is well known to prolong retention of the dosage form in the stomach, often for as long as 12 hours. Because the novel enteric coating compositions release drug in gastric fluid the result is prolonged release of the drug or drugs in the stomach. Multiparticulate leaky enteric compositions also are beneficial to deliver drugs best released in the stomach, especially in a preferred embodiment of dosing at a time proximate to food administration such that food is present with the composition in the stomach.

There is no known method or composition other than as now disclosed herein for use of multiple-unit dosage forms, such as pellets or beads, to improve delivery of drugs that have a window of absorption in the jejunum and/or duodenum.

Thus, it is counterintuitive to deliberately provide a coating that results in prolonged drug input of a therapeutic agent for the purpose of increasing the amount of drug to be absorbed into the body.

It has now been discovered that what has heretofore been considered to be an unacceptable enteric coating composition or inadequate amount of enteric coating material, such as a partial or thin, leaky enteric coat on tablets, capsules or multiparticulates, such as beads or granules, is unexpectedly useful, and also effective to provide an increase in drug delivery for many drugs including those which have an absorption window, i.e., are generally best absorbed from the upper small intestine. Put another way, what have typically been considered non-useful enteric coatings prior to this disclosure are now discovered to be unexpectedly useful to deliver therapeutically active agents.

In one embodiment, an enteric-coated, drug dosage form is deliberately prepared such that the enteric coating composition is “leaky.” A leaky enteric coat allows exposure of the active ingredients to the acid of the stomach and also allows release of a portion of drug from the enteric coat into the stomach fluids. Further, upon passage from the stomach into the upper small intestine the enteric coating material dissolves rapidly such that remaining drug contained within the dosage form is quickly released. Application of this embodiment is particularly useful in formulation of drugs whose bioavailability is often limited due to saturation of absorption processes within the upper small intestine, i.e, an absorption window. Such drugs may be said to have site-specific absorption as discussed above. Further, drug release from the leaky enteric composition generally is too fast to be considered a sustained release (SR) dosage form. But, the result is still a decrease in required frequency of dosing for some absorption window drugs as readily determined by a person of ordinary skill in the art.

In one disclosed embodiment of the present invention, an enteric-coated, drug dosage form is deliberately prepared such that upon contacting gastric fluid, either in vivo or in vitro, the enteric coat is “leaky” in that the enteric coat allows exposure of the active ingredients to acid of the stomach or the in vitro test fluid and also allows release of at least a portion of drug (at least 10%) from the enteric composition into the gastric fluid. Further, upon passage from the stomach into the upper small intestine or transfer into intestinal fluid in vitro, the residual, leaky, enteric coating material dissolves or disintegrates rapidly such that remaining drug (if any) contained within that portion of the dosage form transferred into intestinal fluid is quickly released (at least 60% release of remaining therapeutic in one hour or less upon contacting intestinal fluid).

The enteric material composition may be made leaky by incorporating materials that allow gastric fluid to penetrate into the composition and drug to be released from the composition while the composition is in the stomach. Drug that has been released in the stomach may exert a local effect on the stomach, be absorbed through stomach cells into the blood stream or pass from the stomach into the intestine as drug free of the composition. In a preferred embodiment only a portion of drug in the composition (at least 10%) is released in the stomach and drug still remaining in the composition is rapidly released when the composition passes from the stomach into the intestine.

Particles containing drug, such as beads, granules, and others, are entrapped in a leaky enteric coating to produce an enteric composition that releases drug in gastric fluid. The leaky, enteric-coated particles can be enclosed in a gelatin or other capsule or dosage form that releases the leaky, enteric-coated particles in gastric fluid. In other embodiments, a single-unit dosage form, such as a tablet or capsule, is coated at least partially with a leaky enteric coating. In still other embodiments a matrix tablet or capsule contains enteric compositions such that a portion of drug in the enteric composition (at least 10%) is released in the stomach. Drug still remaining in the composition is rapidly released when the composition passes from the stomach into the intestine.

Embodiments of the disclosed composition may be administered before, during, or soon after a meal such that the food and composition are in the stomach at the same time. Some or all of the composition is retained in the stomach with the food until the food and the composition are emptied, usually by the housekeeper wave. Disclosed embodiments of the composition slowly release drug into gastric fluids for a prolonged period of up to 8 hours or until the drug is all released, or the composition is transported into the intestine and then remaining drug in the composition is rapidly released, preferably in less than one hour in some embodiments. The effect is to extend the time of drug release into the upper intestinal area by up to 10 or more hours, usually up to 7 hours, and more usually up to 4 hours, compared to what occurs with immediate-release dosage forms, and to also provide an earlier release of drug than occurs with known enteric coatings that cause a delay, often of several hours, before drug is released at all. That is, lag time until active ingredient is released from the new enteric compositions is less than two hours, and generally less than one hour, and preferably less than one-half hour when measured in vitro or if measured in vivo.

Another disclosed embodiment releases drug more rapidly after transfer into the intestine than a typical enteric coating. This is thought to occur because the new composition is already partially disrupted, hydrated, and weakened, which results in more rapid dissolution of the new enteric composition, once transferred into the intestine, compared to known compositions.

Drug-containing particulate may be coated with enteric materials, which are either a leaky composition or are a traditional enteric composition that prevents drug release into gastric fluid, said compositions being further treated to produce a leaky enteric composition by any effective method. Such methods include, but are not limited to, compressing the enteric coated particulate into a dosage form, such as tablets, to break or weaken some of the coating(s) such that the resulting novel dosage form releases at least some of the drug in gastric fluid.

Thus, several embodiments of a pharmaceutical formulation comprising an enteric material are disclosed. The embodiments release at least a portion of an active ingredient upon contacting gastric fluid. The remaining portion of the formulation releases active ingredient upon contacting intestinal fluid

A first embodiment of the pharmaceutical composition comprises at least one active ingredient in a core and an enteric coating on the core. The enteric coating further comprises a gastric fluid channeling agent.

Another embodiment of the pharmaceutical composition is designed to provide programmed release of active ingredient: The composition comprises at least one active ingredient, excluding amoxicillin, substantially homogeneously admixed with at least one enteric material comprising a gastric fluid channeling agent.

Still another embodiment of the pharmaceutical composition provides programmed release of active ingredient. The composition comprises at least one active ingredient substantially homogeneously admixed with at least one enteric material comprising a gastric fluid channeling agent. The gastric fluid channeling agent is added in amounts ranging from greater than zero percent to about 400% of the weight of the enteric material.

Still another embodiment of the pharmaceutical composition for providing programmed release of active ingredient comprises at least one active ingredient, excluding amoxicillin, substantially homogeneously admixed with at least one enteric material. The composition delivers at least a portion of the active ingredient upon contacting gastric fluid followed by substantially complete release of active ingredient upon contacting intestinal fluid.

Still another embodiment of the pharmaceutical composition comprises at least one active ingredient, excluding riboflavin, and at least one leaky enteric coating. The composition releases at least 10 percent of the active ingredient mass upon contacting gastric fluid. The remaining active ingredient is released substantially completely after contacting intestinal fluid.

Still another embodiment of the pharmaceutical composition comprises at least one active ingredient, excluding riboflavin. The composition also includes a leaky enteric coating.

Still another embodiment of the pharmaceutical composition comprises at least one active ingredient, excluding amoxicillin, acetyl salicylic acid, bisacodyl, indometacin, riboflavin or sulfamethoxozole. The composition also includes a leaky enteric coating.

Still another embodiment of the pharmaceutical composition consists essentially of a core comprising at least one active ingredient and a leaky enteric coating.

Still another embodiment of the pharmaceutical composition consists essentially of a core comprising at least one active ingredient and an enteric coating. The enteric coating further comprises a gastric fluid channeling agent.

Still another embodiment of the pharmaceutical composition comprises a sugarbead core having at least one active ingredient on or in the core. The composition further comprises an enteric coating comprising a gastric fluid channeling agent.

These and other embodiments of the disclosed composition also have other features or characteristics, or can be used in combination with other features of the invention. For example, disclosed embodiments of the composition generally release at least 10% by mass of the active ingredient in gastric fluid, more typically at least 25%. Still other embodiments provide an active ingredient release profile where at least 10% active ingredient by mass is released in gastric fluid, more typically at least 25%, followed by at least 75% release of remaining active ingredient in one hour or less, with some embodiments releasing at least 75% of remaining active ingredient in 30 minutes or less, upon contacting intestinal fluid. Active ingredient release upon contacting gastric fluid may be zero order, mixed order or first order, followed by substantially immediate release when remaining composition contacts intestinal fluid.

Certain of the embodiments include a gastric fluid channeling agent. For such embodiments, the gastric fluid channeling agent may be hydrophilic, hydrophobic, or a combination of both hydrophilic and hydrophobic. One example of a hydrophilic gastric fluid channeling agent is hydroxylated compounds, such as a sugar, or combinations of sugars. Examples of hydrophobic gastric fluid channeling agents include talc, magnesium salts, silicon dioxide, hydrocarbons, and combinations thereof.

Certain of the embodiments include an enteric coat on or substantially about a core. Such compositions typically have an enteric coating thickness of 25 μm or less, and the coat thickness may be 20 μm or less.

Certain of the embodiments are formulated as a solid composition for oral administration.

Disclosed embodiments of the pharmaceutical composition can comprise one or more additional formulations. Typically, such formulations are designed to provide an active ingredient release profile different from the pharmaceutical composition. For example, a second formulation may provide immediate release in gastric fluid. A specific example of such a composition includes amoxicillin or a biologically active salt thereof as one active ingredient, where the active ingredient of the second formulation is clavulanate or a biologically active salt thereof.

Two or more formulations can be placed in a single capsule or tablet for co-administration. Alternatively, disclosed embodiments of the composition may further comprise an admixture or an overcoat of an immediate release dosage form.

Disclosed embodiments of the pharmaceutical composition may comprise a single active ingredient, or may comprise plural active ingredients. Generally, but not necessarily, the active ingredient has a window of absorption. Examples, without limitation, of active agents having a window of absorption include therapeutic nucleic acids or amino acid sequences, nucleic acids or amino acid derivatives, peptidomimetic drugs, antibiotics, therapeutic ions, vitamins, bronchodilators, anti-gout agents, anti-hypertensive agents, diuretic agents, anti-hyperlipidemic agents or ACE inhibitors, drugs intended for local treatment of the gastrointestinal tract, including anti-tumor agents, histamine (H2) blockers, bismuth salts, synthetic prostaglandins or antibiotic agents, drugs that degrade in the colon, for example metoprolol, formulations useful for treating gastrointestinal associated disorders selected from peptic ulcer, nonulcer dyspepsia, Zollinger-Ellison syndrome, gastritis, duodenitis and the associated ulcerative lesions, stomach or duodenum neoplasms, prazosin, ketanserin, guanabenz acetate, captopril, captopril hydrochloride, enalapril, enalapril maleate, lysinopril, hydralazide, methyldopa, methyldopa hydrochloride, levodopa, carbidopa, benserazide, amlodipine, nitrendipine, nifedipine, nicardipine, verapamil, acyclovir, inosine, pranobex, tribavirine, vidarabine, zidovudine, AZT, active ingredients that exert a medicinal action at the gastric level, including aluminum hydroxide, magnesium carbonate, magnesium oxide, sucralphate, sodium carbenoxolone, pirenzepin, loperamide, cimetidine, ranitidine, famotidine, misoprostol, omeprazol, and combinations thereof Additional examples of active ingredients can be selected from the group consisting of AIDS adjunct agents, alcohol abuse preparations, Alzheimer's disease management agents, amyotrophic lateral sclerosis active ingredient agents, analgesics, anesthetics, antacids, antiarythmics, antibiotics, anticonvulsants, antidepressants, antidiabetic agents, antiemetics, antidotes, antifibrosis active ingredient agents, antifungals, antihistamines, antihypertensives, anti-infective agents, antimicrobials, antineoplastics, antipsychotics, antiparkinsonian agents, antirheumatic agents, appetite stimulants, appetite suppressants, biological response modifiers, biologicals, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfunction active ingredients, fatty acids, gastrointestinal agents, Gaucher's disease management agents, gout preparations, homeopathic remedys, hormones, hypercalcemia management agents, hypnotics, hypocalcemia management agents, immunomodulators, immunosuppressives, ion exchange resins, levocarnitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychoactive ingredient agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, and mixtures thereof.

Still other embodiments of the composition may include an active ingredient selected from the group consisting of abacavir sulfate, abacavir sulfate/lamivudine/zidovudine, acetazolamide, acyclovir, albendazole, albuterol, aldactone, allopurinol, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride, carvedilol, caspofungin acetate, cefazolin, ceftazidime, cefuroxime , chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCl, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

The disclosed pharmaceutical compositions may include an enteric material. Examples, without limitation, of suitable enteric materials include cellulose acetate phthalate (CAP), hydroxypropyl methylcellulose phthalate (HPMCP), polyvinyl acetate phthalate (PVAP), hydroxypropylmethyl cellulose, hydroxypropyl methylcellulose acetate succinate (HPMCAS), cellulose acetate trimellitate, hydroxypropyl methylcellulose succinate, carboxymethyl cellulose, carboxymethyl ethyl cellulose, cellulose acetate phthalate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, polyvinyl acetate phthalate, natural resins such as zein, shellac and copal collophorium, commercially available enteric dispersion systems, including for example Eudragit L30D55, Eudragit FS30D, Eudragit L100, Eudragit S100, Kollicoat EMM30D, Estacryl 30D, Coateric, and Aquateric, and combinations of such materials.

Disclosed embodiments of the pharmaceutical compositions may include other ingredients. For example, and without limitation, such other ingredients include bulking agents, disintegrating agents, anti-adherents and glidants, lubricants, and binding agents. These ingredients are known to persons of ordinary skill in the art. Typical bulking agents include, but are not limited to microcrystalline cellulose (e.g., Avicel®, FMC Corp., Emcocel®, Mendell Incl.), mannitol, xylitol, dicalcium phosphate (eg. Emcompress, Mendell Incl.) calcium sulfate (e.g. Compactrol, Mendell Inc.) starches, lactose, sucrose (Dipac, Amstar, and Nutab, Ingredient Technology), dextrose (Emdex, Mendell, Inc.), sorbitol, cellulose powder (Elcema, Degussa, and Solka Floc, Mendell, Inc.), and combinations thereof. The bulking agent may be present in the composition in any useful amount, which typically ranges from about 5 wt. % to about 90 wt. %, more typically from about 10 wt. % to about 50 wt. %.

Disintegrating agents that may be included in the composition include, but are not limited to, microcrystalline cellulose, starches, crospovidone (e.g., Polyplasdone XL, International Specialty Products.), sodium starch glycolate (Explotab, Mendell Inc.), crosscarmellose sodium (e.g., Ac-Di-Sol, FMC Corp.), and combinations thereof. The disintegrating agent may be present in the composition in any useful amount, which typically is from about 0.5 wt. % to about 30 wt. %, more typically from about 1 wt. % to about 15 wt. %.

Antiadherants and glidants that may be used in the composition include, but are not limited to, talc, corn starch, silicon dioxide, sodium lauryl sulfate, metallic stearates, and combinations thereof. The antiadherant or glidant may be present in the composition in any useful amount, which typically ranges from about 0.2 wt. % to about 15 wt. %, more typically from about 0.5 wt. % to about 5 wt. %.

Lubricants that may be employed in the composition include, but are not limited to, magnesium stearate, calcium stearate, sodium stearate, stearic acid, sodium stearyl fumarate, hydrogenated cotton seed oil (sterotex), talc, and waxes, including but not limited to, beeswax, carnauba wax, cetyl alcohol, glyceryl stearate, glyceryl palmitate, glyceryl behenate, hydrogenated vegetable oils, stearyl alcohol, and combinations thereof. The lubricant may be present in any useful amount, which typically is from about 0.2 wt. % to about 20 wt. %, more typically from about 0.5 wt. % to about 5 wt. %.

Binding agents that may be employed include, but are not limited to, polyvinyl pyrrollidone, starch, methylcellulose, hydroxypropyl methylcellulose, carboxymethyl cellulose, sucrose solution, dextrose solution, acacia, tragacanth, locust bean gum, and combinations thereof. The binding agent may be present in any useful amount, which typically is from about 0.2 wt. % to about 10 wt. %, and more typically from about 0.5 wt. % to about 5 wt. %.

Embodiments of the disclosed composition may increase active ingredient bioavailability at least 20% relative to an immediate release control or a sustained-release formulation control that does not include enteric material. Still other embodiments of the disclosed composition may provide substantially equivalent bioavailability but a reduced active ingredient excretion rate relative to an immediate release control formulation. Still other embodiments of the disclosed composition may provide prolonged drug concentrations for active ingredients having, and even if not having, an absorption window relative to an immediate release control. Certain disclosed embodiments provide controlled in vitro gastric release, followed by pulsatile in vitro intestinal release.

The present disclosure also describes a method for treating a subject having a condition treatable by an active ingredient. The method comprises providing one or more embodiments of the pharmaceutical composition disclosed herein comprising an active ingredient suitable for treating the condition. The pharmaceutical composition is administered to the subject. The active ingredient may be substantially homogeneously mixed with the enteric material. Alternatively, the composition may include a leaky enteric coating, such as a coating comprising a gastric fluid channeling agent or a gastric fluid channel.

The composition may be administered to a fed subject or administered substantially simultaneously when the subject eats or drinks. Alternatively, the composition may be administered to a fasted subject.

A method for making embodiments of the disclosed composition also is described. The method comprises providing a bead core comprising an active ingredient. An enteric material is applied to at least a portion of the bead, and generally on or about a substantial portion of the bead, to form a coat. The composition is then made leaky. This can be accomplished in a number of ways including, without limitation, incorporating a gastric fluid channeling agent, applying pressure, removing solvent, washing, soaking, raising or lowering temperature relative to ambient, abrading, ablating, and any combination thereof.

### BRIEF DESCRIPTION OF THE DRAWINGS

FIG. 1 is a graph of mg. of riboflavin absorbed in people versus time in hours illustrating the cumulative amount of drug absorbed versus time deconvolved from biostudy data for IR (immediate release), SGRD, IGRD, and LGRD (small, intermediate, and large gastric retention device formulation) capsules of Example 1.

FIG. 2 is a dissolution curve of % drug release on the Y-axis as a function of time for a traditional enteric composition coated at 5% weight gain onto drug loaded beads versus new leaky enteric compositions that provide release of drug into gastric fluid at a desired programmed rate, followed by rapid release of drug remaining in the composition when the composition is transferred into intestinal fluid as described in Example 2.

FIG. 3 is a percent of drug dissolved curve versus time in hours at pH 1.4 for two hours and then at pH 6.0 that provides an in vitro dissolution profile of commercial mixed immediate release and enteric-coated pellets of amphetamines, and illustrates that immediate release drug (50%) was released immediately, and that enteric coated drug does not release in gastric fluid but does release when transferred into intestinal fluid as discussed in Example 3.

FIG. 4 is a compartmental diagram that illustrates (i) first-order absorption of drug from immediate release pellets and (ii) zero-order gastric emptying rate in the fed condition of enteric-coated pellets into the intestine and first order absorption of the drug after being released from the pellets as discussed in Example 3.

FIG. 5 is a compartmental diagram that illustrates (i) first-order absorption of drug from immediate release pellets and (ii) first-order gastric emptying rate in the fasted condition of enteric-coated pellets into the intestine and first order absorption of the drug after being released from the pellets.

FIG. 6 provides a mean plot of simulated plasma concentrations versus time for amphetamine from mixed immediate release and enteric-coated pellets in fed subjects, where vertical bars represent standard deviations where the observed values are reported commercial mixed pellets data, and the simulated data are quite accurate.

FIG. 7 is a mean plot of simulated plasma concentrations of amphetamine from mixed immediate release and enteric-coated pellets in fasted subjects, where vertical bars represent standard deviations, and observed values are reported from commercial mixed pellets data.

FIG. 8 is a compartmental diagram of pharmacokinetic model for leaky enteric-coated beads in a fasted condition, where X_{PS} is the amount of drug in beads form in the stomach; X_{SS} is the amount of dissolved drug in the stomach; X_{SI} is the amount of dissolved drug in the intestine; X₁ is the amount of drug in plasma/blood; Dose is a leaky enteric-coated dose; kₑₘ is a first-order rate of drug input into the intestine corresponding to the first-order gastric emptying of beads in fasted condition; kᵣ is a first-order release rate of drug from beads within the stomach; kₛ is a first-order rate of drug input into the intestine corresponding to the first-order gastric emptying of liquid; kₐ is a first-order absorption rate constant of drug; and kₑₗ is a first-order elimination rate constant of drug, as discussed in Example 4.

FIG. 9 is a compartmental diagram of pharmacokinetic model for drug absorption and elimination, and bead transport in the fed condition, using leaky enteric-coated beads, where X_{PS} is the amount of drug in beads form in the stomach; X_{SS} is the amount of dissolved drug in the stomach; X_{SI} is the amount of dissolved drug in the intestine; X₁ is the amount of drug in plasma/blood; Dose is a leaky enteric-coated dose; k₀ is a zero-order rate of drug input into the intestine corresponding to the zero-order gastric emptying of beads in fed condition; kᵣ is a first-order release rate of drug from beads within the stomach; kₛ is a first-order rate of drug input into the intestine corresponding to the first-order gastric emptying of liquid; kₐ is a first-order absorption rate constant of drug; and kₑₗ is a first-order elimination rate constant of drug, as discussed in Example 4.

FIG. 10 illustrates drug concentration in plasma curves for riboflavin vs time following administration to human subjects of (IR) immediate release compared to new leaky enteric compositions as discussed in Example 5.

FIG. 11 illustrates the cumulative amount of hydrochlorthiazide excreted following administration to humans of IRF (immediate release formulation) or GRF (gastric retention formulation) versus time in hours as discussed in Example 8.

FIG. 12 illustrates the rate of urinary excretion of hydrochlorthiazide versus time following administration to humans of IRF (immediate release formulation) or GRF (gastric retention device formulation) versus time in hours as discussed in Example 8.

FIG. 13 illustrates the average rate of urine production following administration to humans of IRF (immediate release formulation) or GRF (gastric retention device formulation) versus time in hours as discussed in Example 8.

FIG. 14 illustrates percent drug release for hydrochlorthiazide from novel leaky enteric coated compositions when tested in gastric fluid for 2 hours followed by transfer into intestinal fluid.

FIG. 15 illustrates drug concentration in plasma curves for hydrochlorothiazide versus time following administration to human subjects of immediate release (IR) compared to new leaky enteric compositions as described in Example 8.

FIG. 16 illustrates percent drug release for ranitidine HCl from some new leaky enteric coated compositions when tested in gastric fluid for 2 hours followed by transfer into intestinal fluid as described in Example 9.

FIG. 17 illustrates percent drug release for ranitidine from some more new leaky enteric coated compositions when tested in gastric fluid for 2 hours followed by transfer into intestinal fluid as described in Example 9.

FIG. 18 illustrates percent drug release for ranitidine from some more new leaky enteric coated compositions when tested in gastric fluid for 2 hours followed by transfer into intestinal fluid as described in Example 9.

FIG. 19 illustrates drug concentration in plasma curves for ranitidine vs time following administration to human subjects of (IR) immediate release compared to new leaky enteric compositions as described in Example 10.

### DETAILED DESCRIPTION

### A. Definitions

Active agent means any therapeutic or diagnostic agent now known or hereinafter discovered that can be formulated as described herein. Examples of therapeutics, without limitation, are listed in Urquhart's U.S. Pat. No. 4,649,043, which is incorporated herein by reference. Additional examples are listed in the American Druggist, p. 21-24 (February, 1995).

Active ingredients includes active agents, therapeutic or diagnostic agents. Active ingredients having an absorption window are known to persons of ordinary skill in the art. For example, U.S. Pat. No. 5,780,057, entitled Pharmaceutical Tablet Characterized by a Showing High Volume Increase When Coming into Contact with Biological Fluids, is primarily concerned with active ingredients that exert their action mostly at the gastroduodenal level and in the first portion of the small intestine. U.S. Pat. No. 6,685,962, entitled Gastroretentive Controlled Release Pharmaceutical Dosage, also concerns drugs with a window of absorption tract. These United States patents are incorporated herein by reference. Examples of drugs having a window of absorption include, but are not limited to, therapeutic nucleic acids or amino acid sequences, nucleic acids or amino acid derivatives, peptidomimetic drugs, antibiotics, therapeutic ions, vitamins, bronchodilators, anti-gout agents, anti-hypertensive agents, diuretic agents, anti-hyperlipidemic agents or ACE inhibitors. The present dosage formulation also may be particularly suitable for the delivery of drugs intended for local treatment of the gastrointestinal tract. Examples of such drugs include, but are not limited to, anti-tumor agents, histamine (H2) blockers, bismuth salts, synthetic prostaglandins or antibiotic agents. The present dosage formulation also may be suitable for the delivery of drugs that degrade in the colon, for example metoprolol. The present dosage formulations are useful for treating gastrointestinal associated disorders selected from peptic ulcer, nonulcer dyspepsia, Zollinger-Ellison syndrome, gastritis, duodenitis and the associated ulcerative lesions, stomach or duodenum neoplasms. Additional specific examples of active ingredients having an absorption window include, without limitation, prazosin, ketanserin, guanabenz acetate, captopril, captopril hydrochloride, enalapril, enalapril maleate, lysinopril, hydralazide, methyldopa, methyldopa hydrochloride, levodopa, carbidopa, benserazide, amlodipine, nitrendipine, nifedipine, nicardipine, verapamil, acyclovir, inosine, pranobex, tribavirine, vidarabine, zidovudine, AZT, active ingredients that exert a medicinal action at the gastric level, including aluminum hydroxide, magnesium carbonate, magnesium oxide, sucralphate, sodium carbenoxolone, pirenzepin, loperamide, cimetidine, ranitidine, famotidine, misoprostol, omeprazol, and combinations thereof. Additional examples of therapeutics, including those having a window of absorption, can be found in the FDA Orange Book, which is incorporated herein by reference. An electronic, searchable version of the Orange Book can be found at http://www.fda.gov/cder/ob/default.htm.

Administration to a subject according to the present invention is intended to be substantially oral administration such that at least a portion of the composition is swallowed.

Channeling agents can be used to tailor drug release from the pharmaceutical composition. Channeling agents provide fluid access to the therapeutic in the pharmaceutical composition in a specific media as desired. The channeling agent may form a tortuous channel in an enteric material by erosion or dissolution of a hydrophobic or hydrophilic material, such as a water soluble, gastric fluid soluble and/or intestinal fluid soluble channeling agent. The channeling agent is incorporated into the enteric material during processing of the dosage form and erodes or leaches from the dosage form after administration of the dosage form to the environment of use. Examples of channeling agents include, without limitation, salts such as sodium chloride and potassium chloride; sugars, such as lactose, sucrose, sorbitol, and mannitol; hydroxylated compounds, including polyvinyl alcohols and glycols, such as polyethylene glycol and propylene glycol; cellulose-derived materials, such as hydroxypropyl cellulose, hydroxypropyl methycellulose, methacrylic acid copolymers; and other miscellaneous materials such as croscarmellose sodium, crospovidone sodium starch glycolate, talc, polyvinyl pyrrolidone, gelling agents such as carbopol, and xanthan gum, or mixtures thereof. The channeling agent also may be a drug that is fluid soluble, including water soluble, gastric fluid soluble, and/or intestinal fluid soluble. The channeling agent is included in the dosage form in an amount to allow active ingredients to leak through the enteric material in gastric fluid, with the preferred amounts being selected to achieve the desired result. Such amounts typically range from greater than 0% to about 400% of the total weight of the enteric material. For coatings, such amounts range from greater than 0% to about 100%, and even more typically from about 5% to about 40% of the total weight of the enteric material. For substantially homogeneous admixtures, channeling agent amounts typically range from about 25% to about 350%, and even more typically from about 75% to about 250% of the total weight of the enteric material.

Coating and overcoating are used interchangeably herein and refer to applying at least one coat, and perhaps plural coats, over a core compact, and core compact or core as used herein.

Controlled release includes timed release, sustained release, extended release, pulse release, prolonged release and other such terms which describe a sustained release pattern from dosage forms as is known to a person of ordinary skill in the art and does not include immediate release, delayed release, or programmed release as described herein. It is common, for one example, to identify a formulation as sustained release if dissolution of the active agent during in vitro dissolution tests known to those of ordinary skill in the art is slower than dissolution of the same active agent when compared to an immediate release control formulation. The cause of the slower dissolution is utilization of a type of formulation or process that is known by those or ordinary skill in the art to provide sustained release of active agents. In dissolution tests, generally it is preferred for sustained release formulations that it takes longer than 3 hours for 65% dissolution of the active ingredient, more preferred that it takes longer than 4 hours for 75% dissolution of the active ingredient, and even more preferred that it takes longer than 7 hours for 75% dissolution of the active ingredient. In some cases, it may take more than 18 hours for 85% dissolution of the active ingredient. Some types of sustained-release formulations, without limitation, include formulations known as osmotic pump tablets or capsules, hydrophilic or other polymer or wax matrix tablets, beads, or capsules, and diffusional release controlling membrane containing dosage forms.

Core refers to the center portion of a layered or coated drug delivery system. The core portion typically comprises active agent(s), either with or without added excipients, and also includes beads, such as sugar beads or extruded beads, tablets, beads, particles, or capsules impregnated or coated with an active agent.

Diagnostic means, without limitation, a material useful for testing for the presence or absence of a material or disease, and/or a material that enhances tissue or cavity imaging.

Dissolution or release of drug into gastric fluid includes release into gastric fluid of the stomach in vivo or during in vitro testing. Many such tests are known in the art. One such test, for example, is the United States Pharmacopea (U.S. Pharmacopeia, 23, U.S. Pharmacopeial Convention: Rockville, Md., 1994, pp. 1795) test for drug release for enteric-coated dosage forms.

An effective amount is an amount of a diagnostic or therapeutic agent that is useful for producing a desired effect.

An enteric composition is a delayed release composition that prevents release of active agent in gastric fluid or exposure of active agent to gastric fluid while the enteric composition is in the stomach or in gastric fluid in an in vitro dissolution test, and then the active agent is released from the enteric composition or that portion of the enteric composition that is transferred into the intestine, such as enteric-coated multiparticulates, for example, where some enteric-coated particles are transferred into the intestine while others remain in the stomach, or into an in vitro dissolution test in neutral medium (e.g., pH 6.8 to 8.0). Examples of enteric materials include, but are not limited to, cellulose acetate phthalate (CAP), hydroxypropyl methylcellulose phthalate (HPMCP), polyvinyl acetate phthalate (PVAP), hydroxypropylmethyl cellulose, hydroxypropyl methylcellulose acetate succinate (HPMCAS), cellulose acetate trimellitate, hydroxypropyl methylcellulose succinate, carboxymethyl cellulose, carboxymethyl ethyl cellulose, cellulose acetate phthalate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, polyvinyl acetate phthalate, natural resins such as zein, shellac and copal collophorium, commercially available enteric dispersion systems, including for example Eudragit L30D55, Eudragit FS30D, Eudragit L100, Eudragit S100, Kollicoat EMM30D, Estacryl 30D, Coateric, and Aquateric, and combinations of such materials.

Gastric fluid as used herein means the endogenous fluid medium of the stomach, including water and secretions, or simulated gastric fluid, or other aqueous fluids of pH less than 5.5 that are useful to measure drug dissolution from an enteric formulation.

Immediate release means 80% or more of the active ingredient is released when in unprotected contact with gastric fluid or intestinal fluid within 30 minutes of exposure to such fluid.

Intestinal fluid is endogenous fluid medium of the intestine, including water and secretions, or simulated intestinal fluid, or other aqueous fluids of pH 5.5 or greater that are useful for measuring drug dissolution from an enteric coated product.

Leaky enteric composition is an enteric composition that has been modified by formulation, process, or method so that the composition does release active ingredient in gastric fluid or when exposed to gastric fluid. Typically, more than about 5%, even more typically 10% or greater, of the active ingredient is released while the enteric composition is in the stomach or in gastric fluid in an in vitro dissolution test, and then the active ingredient is released from the composition or that portion of the composition that is transferred into the intestine or into an in vitro dissolution test in neutral medium (e.g., pH 6.8 up to 8.0). Leaky enteric composition includes any composition that comprises a pH-sensitive pharmaceutical excipient that has relatively low solubility in gastric fluid and relatively higher solubility (is at least 4 times more soluble) in neutral medium (pH 6.8 up to pH 8.0), and the composition allows release in gastric fluid or exposure to gastric fluid of more than about 5% of the active ingredient, and even more typically greater than about 10%, while the enteric composition is in the stomach or in gastric fluid in a in vitro dissolution test, and then at least 75% of any active ingredient not released in gastric fluid is released from the composition or portion of the composition that is transferred into the intestine or into an in vitro dissolution test in neutral medium (e.g., pH 6.8 up to 8.0).

Other ingredients include, for example, bulking agents, disintegrating agents, anti-adherents and glidants, lubricants, binding agents, flavoring agents, etc., including without limitation: bulking agents, such as microcrystalline cellulose (e.g., Avicel®, FMC Corp., Emcocel®, Mendell Incl.), mannitol, xylitol, dicalcium phosphate (eg. Emcompress, Mendell Incl.) calcium sulfate (e.g. Compactrol, Mendell Inc.) starches, lactose, sucrose (Dipac, Amstar, and Nutab, Ingredient Technology), dextrose (Emdex, Mendell, Inc.), sorbitol, cellulose powder (Elcema, Degussa, and Solka Floc, Mendell, Inc.), and combinations thereof; disintegrating agents, such as microcrystalline cellulose, starches, crospovidone (e.g., Polyplasdone XL, International Specialty Products.), sodium starch glycolate (Explotab, Mendell Inc.), crosscarmellose sodium (e.g., Ac-Di-Sol, FMC Corp.), and combinations thereof; antiadherants and glidants, such as talc, corn starch, silicon dioxide, sodium lauryl sulfate, metallic stearates, and combinations thereof; lubricants, such as magnesium stearate, calcium stearate, sodium stearate, stearic acid, sodium stearyl fumarate, hydrogenated cotton seed oil (sterotex), talc, and waxes, including but not limited to, beeswax, camauba wax, cetyl alcohol, glyceryl stearate, glyceryl palmitate, glyceryl behenate, hydrogenated vegetable oils, stearyl alcohol, and combinations thereof; and binding agents, such as polyvinyl pyrrollidone, starch, methylcellulose, hydroxypropyl methylcellulose, carboxymethyl cellulose, sucrose solution, dextrose solution, acacia, tragacanth, locust bean gum, and combinations thereof.

Programmed release means release or exposure of active ingredient in gastric fluid at a rate slower than immediate release, that is less than 80% release on exposure to gastric fluid within 30 minutes, followed by release of more than 60% of active agent not yet released or exposed in gastric fluid in less than one hour from the composition or that portion of the composition when it is transferred into intestinal fluid.

Simulated gastric fluid means any fluid that is generally recognized as providing a useful substitute for authentic gastric fluid in experiments designed to assess the chemical, biochemical or dissolution behavior of substances in the stomach. One such simulated gastric fluid is USP gastric fluid TS, without enzymes, United States Pharmacopeia and National Formulary 24/19 p. 2235 (1999). Thus, it will be understood throughout this disclosure, unless noted otherwise, that “gastric fluid” means any gastric fluid including authentic gastric fluid or simulated gastric fluid.

Simulated intestinal fluid means any fluid that is generally recognized as providing a useful substitute for authentic intestinal fluid in experiments designed to assess the chemical or biochemical or dissolution behavior of substances in the intestines. One such simulated intestinal fluid is USP intestinal fluid TS, without enzymes, United States Pharmacopeia and National Formulary 24/19 (1999). Thus, it will be understood throughout this disclosure that “intestinal fluid” means any intestinal fluid including authentic intestinal fluid or simulated intestinal fluid.

Spheres, millispheres, pellets, granules, beads, multiparticulates, and particulates are terms which are interchangeable when referring to the drug delivery systems of this invention.

Tablet is a term known to persons of ordinary skill in the art, and is used herein to include all such compacted, or molded, or otherwise formed materials without limitation in terms of sizes or shapes, and all methods of preparation. Thus, as one common example, compressed or molded shapes which are known as caplets, are included. Plural pellets can be compacted into tablets, and such tablets may be chewable.

### B. EXAMPLES

The following examples are provided to illustrate certain features of disclosed embodiments. The scope of the present invention should not be limited to those features illustrated.

### Example 1

This example shows that if a drug with an absorption window can be delivered into the upper intestine from the stomach via controlled release in gastric fluid, then bioavailability of the drug can be increased by more than 20% and prolonged drug concentrations in the body occur compared to orally administering an immediate release drug formulation as shown in my European Patent Application PCT WO 03/015745 A1.

The drug riboflavin was formulated in a gastric retention formulation to provide controlled drug release in gastric fluid and thereby prolonged drug input from the stomach into the intestine so long as the device was in the stomach and still contained drug, and administered in biostudies, and compared to administration of immediate release riboflavin as shown in my European Patent Application PCT WO 03/015745 A1.

Relative fractional absorption of riboflavin from different riboflavin containing formulations was evaluated from urinary excretion data. Mean pharmacokinetic parameters for the different treatments are shown in the following table.

TABLE 1
Pharmacokinetic Parameters of Riboflavin after Oral Administration of
100 mg in Immediate Release or Slow Input from GRD Capsules to
Fasted Volunteers.

| | Treatments | | | |
|---|---|---|---|---|
| | (IR) | (SGRD) | (IGRD) | (LGRD) |
| Recovery from | 5.33 ± 1.74 | 4.09 ± 1.67 | 9.3 ± 5.27 | 17.36 ± 9.7 |
| 0-24 h (mg) | | | | |
| Max. Urinary | 1.36 ± 0.42 | 1.14 ± 0.59 | 2.05 ± 0.99 | 2.52 ± 0.98 |
| excretion rate | | | | |
| (mg/h) | | | | |
| Time of max. | 2.5 ± 0.6 | 2.33 ± 0.97 | 3.25 ± 1.1 | 5.08 ± 2.4 |
| excretion | | | | |
| rate (h) | | | | |
| Mean | 4.73 ± 0.83 | 5.98 ± 1.06 | 5.27 ± 1.7 | 6.99 ± 1.18 |
| Residence | | | | |
| time (h) | | | | |

Data are mean values ± SE

IR is immediate release from a commercial product; SGRD is a small gastric retention device/formulation; IGRD is an intermediate size gastric retention device/formulation: LGRD is a large gastric retention device/formulation (see European Patent Application PCT WO 03/015745 A1.

Mean drug recovery ₀₋₂₄ₕ in subjects urine from the LGRD controlled drug release in gastric fluid (17.3mg) was determined to be 3.25 times (and statistically significantly (P<0.05) different relative to the mean) the mean drug recovery₀₋₂₄ₕ in subjects urine from the IR capsule (5.33 mg). Statistical comparison of Rₘₐₓ and Tₘₐₓ parameters also indicated a significant difference (P<0.05) between results from slow drug input from the LGRD capsule (2.5±0.98 mg/h and 5.08±2.4 hr respectively) and the IR capsule (1.36±0.4 mg/h and 2.5±0.63 hr respectively). Improved bioavailability of riboflavin from slow input of drug from the stomach into the intestine resulted because the released drug passed gradually through the absorption window and more efficient absorption occurred. Formulation of riboflavin into an embodiment of a leaky, enteric-coated bead formulation of the instant invention also results in an increase in bioavailbility when compared to drug bioavailability from an immediate release formulation because of the unique drug release pattern from the leaky, enteric-coated formulation as described elsewhere herein.

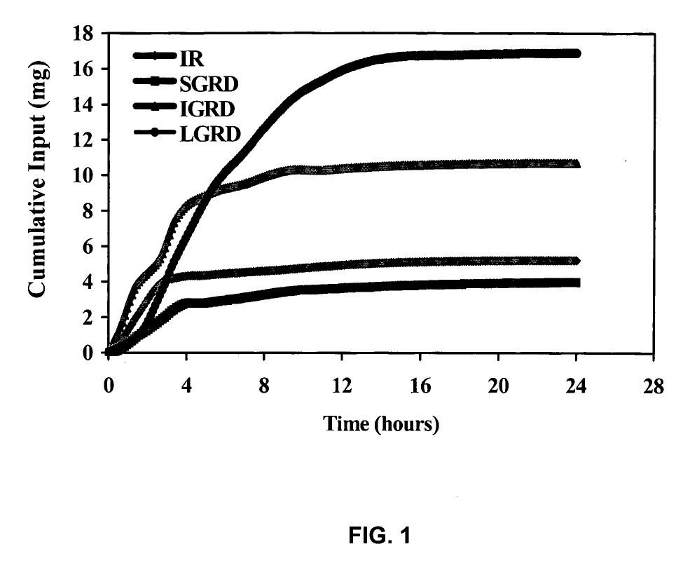

FIG. 1 shows the cumulative amount of drug absorbed versus time deconvolved from biostudy data for the IR, SGRD, IGRD, and LGRD capsules. Absorption continued for up to about 15 hours for the slow drug input LGRD capsule formulation before it stopped. This suggests that the LGRD stayed in the stomach and slowly released the drug for about 15 hours. Drug absorption from the slow release SGRD capsule continued only for 3 hours, indicating that the device was emptied from the stomach by the housekeeper wave (due to its small size) as rapidly as the IR dose. Thus, even though this formulation provided sustained drug release in gastric fluid, when the drug was trapped inside a composition that provided sustained drug release in intestinal fluid and that passed by the absorption window before drug was released, then the bioavailability of the drug was decreased. That is why drugs with absorption windows cannot be formulated using traditional means to provide useful prolonged or sustained drug input.

For compositions of embodiments of the new leaky enteric formulations described herein, some of the drug in the leaky enteric formulation is programmed released in the stomach gastric fluid and trickles into the intestine while the dosage form remains in the stomach. This released drug is well absorbed just as is demonstrated in the GRF example during the time the GRF is in the stomach. Then, any drug that remains inside the dosage form is rapidly released, preferably more than 60% of active agent not yet released or exposed in gastric fluid is released in less than one hour from the composition or that portion of the composition when the new leaky enteric formulation or a portion thereof is transferred into the intestine. Thus, embodiments of the novel leaky enteric formulations disclosed herein avoid the case shown above for the slow release GRF example where bioavailability is reduced relative to an immediate-release formulation if drug is retained inside the slow-release, GRF formulations when the GRF is transferred from the stomach into the intestine, and passes the absorption window. Note that improved bioavailability of riboflavin from the LGRD capsule was more than triple that measured after administration of the IR formulation in this study.

### Example 2

Spray-Coating Procedures

Nonpareil sugar beads 18-20 mesh (approximately 0.8 mm in diameter) were placed into a coating chamber of a fluid-bed spray coater (Niro-Aeromatic, model STREA-1, Niro-Aeromatic, Ltd.) with a Wurster column insert. The Wurster column was approximately 1 inch away from the bottom screen of the coating chamber. The sugar beads were fluidized for 5 minutes to equilibrate with the coating temperature (40-45° C.) before starting the coating process. At the end of each coating step, the coated beads were dried in the coating chamber at 40° C. for approximately 10-15 minutes. Model drugs and leaky enteric-coating polymers were sprayed onto sugar beads (batch size 40-200 g) according to the drug being formulated.

All coating solutions or dispersions were continuously delivered through a feeding tube by a peristaltic pump (Rabbit Peristaltic pump, Gilson Electronics, Middleton, Wis.). Coating solutions or dispersions were kept stirring using a magnetic stirrer to ensure homogeneity of the solution or dispersions. For each coating step, coating conditions were monitored and adjusted to maintain effective coating conditions. After each coating step, beads were sieved to remove agglomerated and fine particles before proceeding to the next steps.

A composition of enteric coating polymer material (Eudragit L30D-55 from Rhome Pharma) in a quantity that prevents more than 5% drug release in gastric fluid for the beads used was prepared with lactose to modify the coating so as to produce a coating material that releases drug in gastric fluid.

TABLE 2
Leaky, Enteric-Coated Beads Formulations of Riboflavin-5-Phosphate

| | | Amount of Leaky |
|---|---|---|
| | Composition of Leaky | Enteric-Coating Polymer |
| Formulations | Enteric-Coating Polymer | (% of Drug-Loaded Beads)^{b} |
| RF1 | EUD^{a} | 5% |
| RF2 | EUD with 50% lactose | 5% |
| RF3 | EUD with 65% lactose | 5% |
| RF4 | EUD with 83.5% lactose | 5% |

^{a}Eudragit ® L30D-55 (EUD). Working titles of coating formulation are used here and in the figures. Final coating formulations given below.
^{b}Weight gain of beads coated with the coating material after drying the coated beads.

TABLE 3
Ingredients of Leaky Enteric-Coating Composition of
Riboflavin-5-Phosphate Formulations.

| | |
|---|---|
| | Formulations |
| Ingredients | (% of Total Coating Materials) |

| (Solid Composition) | RF1 | RF2 | RF3 | RF4 |
|---|---|---|---|---|
| Eudragit ® L30D-55 | 66.7 | 50.0 | 46.5 | 42.8 |
| Talcum | 33.3 | 25.0 | 23.3 | 21.4 |
| Lactose | — | 25.0 | 30.2 | 35.8 |

### Compositions and Preparations of Coating Solution/Dispersion for Riboflavin-5-Phosphate

TABLE 4
Riboflavin loading solution

| | | |
|---|---|---|
| Riboflavin-5-phosphate | 7.5 | g |
| PVP K-30 | 2.0 | g |
| Hydroxypropyl cellulose (HPC) EXF | 1.0 | g |
| Deionized water | 250.0 | ml |

Accurately weighed HPC EXF was dispersed in 50 ml of hot deionized water. Cool deionized water was added to the well-dispersed HPC and the solution was stirred until clear. PVP K-30 was then added and well mixed. Finally, riboflavin was added to the solution and stirred until dissolved. Loading solution was kept protected from light throughout this process.

TABLE 5
Eudragit ® L30D-55 dispersion (EUD)

| | | |
|---|---|---|
| Eudragit ® L30D-55 | 50.0 | g |
| Triethyl citrate | 1.5 | g |
| Talcum | 7.5 | g |
| Deionized water | 50.0 | ml |

Eudragit® L30D-55 was accurately weighed into a beaker. Triethyl citrate was added to Eudragit® suspension and gently mixed. Talcum was dispersed in deionized water. The talcum dispersion was then added into Eudragit® mixture and gently mixed. This mixture was gently stirred continuously.

TABLE 6
Eudragit ® L30D-55 with 50% lactose dispersion

| | | |
|---|---|---|
| Eudragit ® L30D-55 | 50.0 | g |
| Triethyl citrate | 1.5 | g |
| Talcum | 7.5 | g |
| Lactose | 7.5 | g |
| Deionized water | 125.0 | ml |

Accurately weighed lactose was dissolved in 75 ml of deionized water (solution may be warmed to facilitate the dissolution). Talcum was dispersed in the remaining deionized water. Talcum dispersion was added to lactose solution and kept stirring. Eudragit® L30D-55 was accurately weighed into a beaker. Triethyl citrate was added to Eudragit® suspension and gently mixed. The lactose and talcum dispersion was then added into Eudragit® mixture and gently mixed with continuous stirring. The amount of lactose used in studied formulations was calculated as a percentage of Eudragit® polymer solid (Eudragit® polymer suspension contains 30% polymer solid). The volume of deionized water varied as needed to sufficiently dissolve lactose for other lactose formulations (generally, one part of lactose can be dissolved in 10 part of water).

### In Vitro Dissolution Testing of Studied Formulations

In vitro drug release profiles of studied formulations were obtained using the United States Pharmacopoeia (USP) XXIII dissolution apparatus I, basket method (VK 7000, Vankel Industries, Inc., Cary, N.C.). Dissolution was studied at a basket rotation speed of 100 rpm and the dissolution bath temperature was maintained at about 37.5° C. Dissolution testing of all formulations was performed in triplicate.

Studied formulations were placed into dissolution baskets, which were then immersed in dissolution vessels containing 600 ml of simulated gastric fluid. Dissolution testing was run in simulated gastric fluid for 2 hours. At the end of the 2-hour period, the dissolution baskets were transferred into phosphate buffer pH 6.0. Dissolution testing was continued in phosphate buffer until studied formulations were completely disintegrated.

Five (5) ml samples were manually collected without medium replacement at 0.17, 0.33, 0.5, 0.75, 1, 2, 2.08, 2.17, 2.25, 2.5, 2.75, 3, 4 and 5 hours. The samples were centrifuged at 3,000 rpm for 20 minutes. Supernatant was then collected and measured by UV spectrophotometer at 445, 330, and 318 nm for riboflavin, ranitidine, and hydrochlorothiazide, respectively. The amount of drug released was determined using an appropriate standard curve.

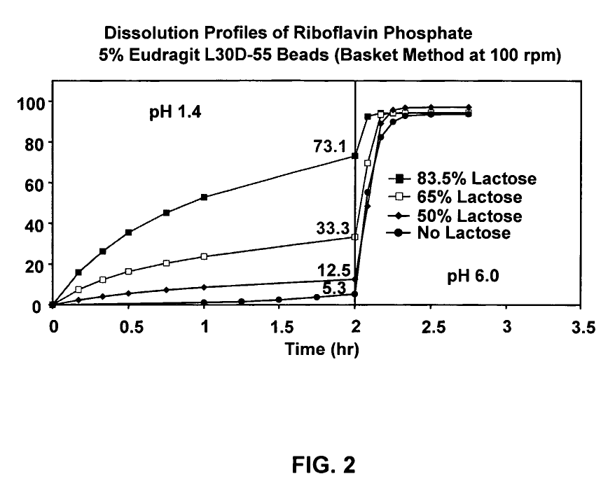

Average drug releases and their standard deviations were calculated from three replications in all dissolution experiments. Dissolution profiles are presented in FIG. 2 as percent drug release versus time curves.

FIG. 2 shows % active ingredient (riboflavin) release on the Y-axis and exemplifies performance of new leaky enteric compositions to provide release of drug into gastric fluid at desired programmed rates, followed by rapid release (more than 60% in less than one hour in this case) of drug remaining in the composition when the composition is transferred into intestinal fluid. The traditional enteric composition with no lactose did not release measurable drug in gastric fluid during the first hour and only released 5.3% of active ingredient after two hours in gastric fluid. The 5.3% drug release in gastric fluid in two hours satisfies USP requirements for enteric-coated (delayed release) dosage forms.

A coating also can be made to provide programmed release of active ingredient in gastric fluid by making the coat too thin to effectively prevent drug release in gastric fluid. That is, just as the traditional enteric coating without lactose can be made thicker to prevent drug release in two hours in the dissolution test in gastric fluid, so also can the same coating be provided in a thinner layer to become a leaky enteric coating and provide desired programmed release of active ingredient in gastric fluid. Increasing weight gain of the coating generally results in increasing coating thickness and resistance to active ingredient release in gastric fluid, wherease decreasing weight gain of the coating generally results in decreasing coating thickness and increased active ingredient release in gastric fluid. In some cases, it is preferable to make the coating thicker to obtain more consistent results and a greater ability to program drug release in gastric fluid. In these cases a hydrophilic or hydrophobic additive that promotes drug release in gastric fluid is generally included in the new leaky enteric compositions.

### Example 3

Pharmacokinetic models have been used to simulate data for drug concentration versus time curves following administration of a 50/50 mixture of immediate release drug and enteric-coated pellets that do not release drug in gastric fluid. A preliminary report indicated the data are quite accurate (Prapoch Watanalumlerd, J. Mark Christensen, and James W. Ayres, Gastrointestinal Transit Effect on Drug Pharmacokinetics from Mixed Immediate Release and Enteric-coated Amphetamine Beads, Poster presentation at American Association of Pharmaceutical Scientists (AAPS) Annual Meeting, Nov. 10-14, 2002, Toronto, Canada). This example now shows model equations and assumptions, and that pharmacokinetic models used to generate data for drug concentration versus time curves provide very good data when applied to enteric-coated beads mixed with immediate release drug that is not enteric coated. In this case, data for drug concentrations versus time in fed and fasted subjects are taken from a product based on U.S. Pat. No. 6, 322,819. The formulation contained 50% of the dose available as immediate release drug and 50% of the dose in enteric coated pellets that did not release any drug until the pellets passed out of the stomach and were in the intestine.

Commercial mixed immediate-release and enteric-coated amphetamine capsules (Adderall XR™) containing 10 mg of immediate-release amphetamine salts and 10 mg of delayed-release (enteric coated) amphetamine salts were used for in vitro dissolution test. Amphetamine dissolution profile was obtained using USP dissolution apparatus II at 37.5° C. and paddle speed 100 rpm. The formulation without capsule shell was run in simulated gastric fluid (pH 1.4) for 2 hours before the dissolution medium was adjusted to pH 6.0 by adding alkaline solution (0.2 M tribasic sodium phosphate solution) and de-ionized water. Samples were assayed for amphetamine concentration using an ultraviolet spectrophotometer at 257 nm.

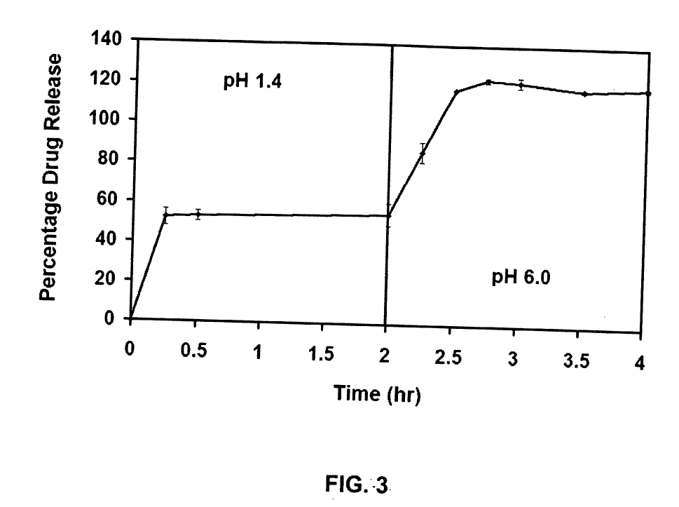

FIG. 3 confirms that immediate release drug was released immediately, and that enteric-coated drug does not release in gastric fluid but does release when transferred into intestinal fluid.

A. Pharmacokinetic Models

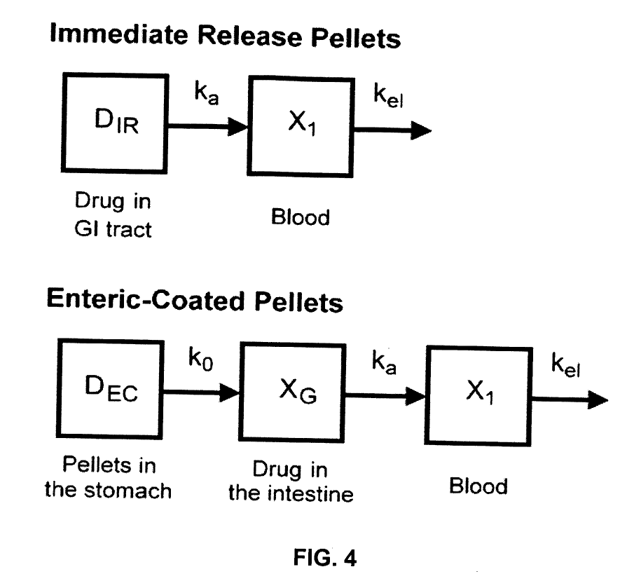

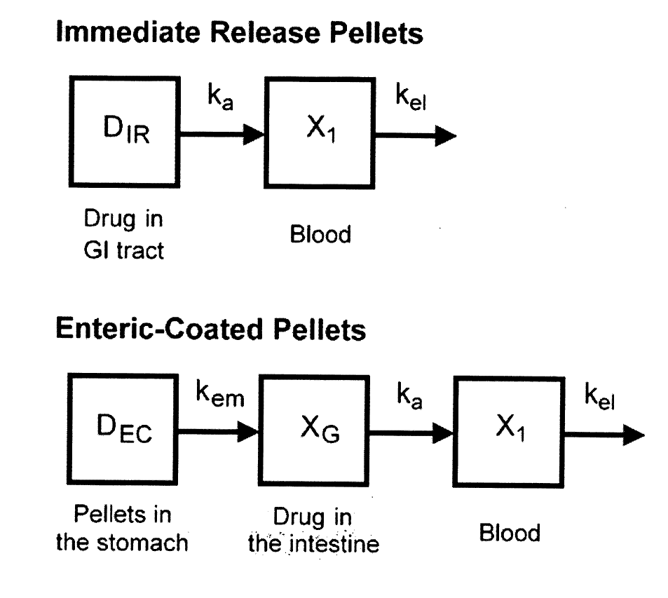

Using knowledge about gastric emptying and GI transit, compartmental diagrams for pharmacokinetics of drugs from mixed immediate release and enteric-coated pellets in the fed and fasted condition are created and shown in FIGS. 4 and 5, respectively. Compartmental diagrams in FIG. 4 represent (i) first-order absorption of drug from immediate release pellets and (ii) zero-order gastric emptying rate in the fed condition of enteric-coated pellets into the intestine, and first order absorption of the drug after being released from the pellets. Compartmental diagrams in FIG. 5 represent (i) first-order absorption of immediate release pellets and (ii) first-order gastric emptying rate in the fasted condition of enteric-coated pellets into the intestine, and first order absorption of the drug after being released from the pellets. These compartmental diagrams apply to drugs when pharmacokinetics following oral administration can be described by a one-compartment model.

With reference to FIG. 4, a compartmental diagram of pharmacokinetic models for mixed, immediate-release and enteric-coated pellets in fed condition, X_{G} is the amount of released drug in the intestine; X₁ is the amount of drug in plasma/blood; D_{IR} is an immediate release dose; D_{EC} is an enteric-coated dose; k₀ is a zero-order input of drug corresponding to the zero-order gastric emptying of enteric-coated pellets in fed condition; kₐ is a first-order absorption rate constant of drug; and kₑₗ is a first-order elimination rate constant of drug.

With reference to FIG. 5, a compartmental diagram of pharmacokinetic model for mixed, immediate-release and enteric-coated pellets in fasted condition, X_{G} is the amount of released drug in the intestine; X₁ is the amount of drug in plasma/blood; D_{IR} is an immediate release dose; D_{EC} is an enteric-coated dose; kₑₘ is a first-order rate of drug input corresponding to the first-order gastric emptying of enteric-coated pellets in fasted condition; kₐ is a first-order absorption rate constant of drug; and kₑₗ is a first-order elimination rate constant of drug.

Pharmacokinetic models describing pharmacokinetics of mixed immediate release and enteric-coated pellets in the fed condition are presented in Equations 1-3.

For fed condition:

$$\begin{matrix}When t \leq τ , \\ {C}_{1} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{0}}{V \cdot {k}_{el}} [ 1 - \frac{{k}_{el}}{( {k}_{el} - {k}_{a} )} {ⅇ}^{- {k}_{a} t} - \frac{{k}_{a}}{( {k}_{a} - {k}_{el} )} {ⅇ}^{- {k}_{el} t} ] & Eqn 1 \\ When t > τ , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{0}}{V \cdot {k}_{el}} [ 1 - \frac{{k}_{el}}{( {k}_{el} - {k}_{a} )} {ⅇ}^{- {k}_{a} τ} - \frac{{k}_{a}}{( {k}_{a} - {k}_{el} )} {ⅇ}^{- {k}_{el} τ} ] {ⅇ}^{- {k}_{el} ( t - τ )} + \frac{{k}_{0} ( 1 - {ⅇ}^{- {k}_{a} τ} )}{V ( {k}_{a} - {k}_{el} )} [ {ⅇ}^{- {k}_{el} ( t - τ )} - {ⅇ}^{- {k}_{a} ( t - τ )} ] & Eqn 2\end{matrix}$$

For the fasted condition, pharmacokinetic model describing plasma concentration of a drug from mixed, immediate-release and enteric-coated pellets can be obtained by combining the pharmacokinetic model of oral-controlled, first-order-release dosage form with a typical extravascular pharmocokinetic model for immediate release pellets. This model is presented in Equation 3.

For a fasted condition:

$${C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{em} {k}_{a} {D}_{EC}}{V} [ \frac{{ⅇ}^{- {k}_{em} t}}{( {k}_{a} - {k}_{em} ) ( {k}_{el} - {k}_{em} )} + \frac{{ⅇ}^{- {k}_{a} t}}{( {k}_{em} - {k}_{a} ) ( {k}_{el} - {k}_{a} )} + \frac{{ⅇ}^{- {k}_{el} t}}{( {k}_{em} - {k}_{el} ) ( {k}_{a} - {k}_{el} )} ] \qquad Eqn 3$$

Cₜ is plasma concentration of the drug at time t. D_{IR} is an immediate release dose. D_{EC} is an enteric-coated dose. kₑₘ represents a first-order rate of drug input corresponding to the first-order gastric emptying of enteric-coated pellets in fasted state. k₀ represents a zero-order input of drug corresponding to the zero-order gastric emptying of enteric-coated pellets in fed state. kₐ and kₑₗ represent a first-order absorption rate constant and a first-order elimination rate constant of drug, respectively. Tau (τ) is gastric emptying time of enteric-coated pellets (i.e. the time of zero-order input). V is an apparent volume of distribution for the blood compartment. These equations may be multiplied by a factor F, which is the fraction of absorbed drug.

Since a gastric emptying lag time is expected and will affect drug release from enteric-coated pellets, the above equations are modified by including another time parameter−lag time of emptying (lag), as presented in Equations 4-8.

For fed condition:

$$\begin{matrix}When t \leq lag , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) & Eqn 4 \\ When lag < t \leq τ + lag , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{0}}{V \cdot {k}_{el}} [ 1 - \frac{{k}_{el}}{( {k}_{el} - {k}_{a} )} {ⅇ}^{- {k}_{a} ( t - lag )} - \frac{{k}_{a}}{( {k}_{a} - {k}_{el} )} {ⅇ}^{- {k}_{el} ( t - lag )} ] & Eqn 5 \\ When t > τ + lag , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{0}}{V \cdot {k}_{el}} [ 1 - \frac{{k}_{el}}{( {k}_{el} - {k}_{a} )} {ⅇ}^{- {k}_{a} τ} - \frac{{k}_{a}}{( {k}_{a} - {k}_{el} )} {ⅇ}^{- {k}_{el} τ} ] {ⅇ}^{- {k}_{el} ( t - lag - τ )} + \frac{{k}_{0} ( 1 - {ⅇ}^{- {k}_{a} t} )}{V ( {k}_{a} - {k}_{el} )} [ {ⅇ}^{- {k}_{el} ( t - lag - τ )} - {ⅇ}^{- {k}_{a} ( t - lag - τ )} ] & Eqn 6\end{matrix}$$

For fasted condition:

$$\begin{matrix}When t \leq lag , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) & Eqn 7 \\ When t > lag , \\ {C}_{t} = \frac{{k}_{a} {D}_{IR}}{V ( {k}_{a} - {k}_{el} )} ( {ⅇ}^{- {k}_{el} t} - {ⅇ}^{- {k}_{a} t} ) + \frac{{k}_{em} {k}_{a} {D}_{EC}}{V} [ \frac{{ⅇ}^{- {k}_{em} ( t - lag )}}{( {k}_{a} - {k}_{em} ) ( {k}_{el} - {k}_{em} )} + \frac{{ⅇ}^{- {k}_{a} ( t - lag )}}{( {k}_{em} - {k}_{a} ) ( {k}_{el} - {k}_{a} )} + \frac{{ⅇ}^{- {k}_{el} ( t - lag )}}{( {k}_{em} - {k}_{el} ) ( {k}_{a} - {k}_{el} )} ] & Eqn 8\end{matrix}$$

Model Assumptions

Several assumptions were made for the models presented above, including:

1. Pharmacokinetic of the drug involved is linear in the dosing range of interest. Thus, superposition for determination of plasma drug concentrations can be applied.

2. Enteric-coated portion of formulations is in multiple-unit pellet/granule (multi-particulate) form and does not release drug in gastric fluid.

3. Enteric-coated polymer dissolves instantaneously upon transfer into the intestine.

4. After the pH-dependent polymer on enteric-coated pellets dissolves in the intestine, then drug release is instantaneous.

5. Once being released from formulations, the drug is absorbed from the gastrointestinal tract by a first-order process.

6. Pharmacokinetics of the drug after absorption is well described by a one-compartment open model.

7. The elimination process is a first-order process.

No assumptions are required to be exact, or even correct, so long as the model adequately approximates the processes described. Results below clearly support validity of the assumptions and models.

Monte Carlo Simulations

Pharmacokinetic models above were used in Monte Carlo simulations of plasma concentration-time curves from mixed immediate release and enteric-coated pellets of amphetamine. Five hundred trials for each simulation were performed using Crystal Ball 2000.2 software (Decisioneering, Inc., Denver, Colo.). The simulated plasma data concentration-time curves of amphetamine are presented as a mean plot (along with its standard deviation). The peak plasma concentration (Cₘₐₓ) of the actual data is then compared to Cₘₐₓ of simulated data.

Model Parameters

Following oral administration of immediate-release amphetamine, a one-compartment model best describes plasma drug concentrations both in adults and children. Pharmacokinetic parameters of amphetamine used in the simulations were obtained from pharmacokinetic fitting of available plasma concentration data of amphetamine (Sifton, D. W. Physician Desk Reference. 57th Ed. Thomson PDR: Montvale, N.J., 2003.) using Kinetica 2000 software, version 3.0 (InnaPhase Corporation, Philadelphia, Pa.). These parameters are volume of distribution divided by fraction of dose absorbed (V/F), absorption rate constant (kₐ), and elimination rate constant (kₑₗ). Since pharmacokinetics of d-amphetamine and 1-amphetamine are similar, only simulations of d-amphetamine will be carried out. Pharmacokinetic parameters of d-amphetamine used in the simulations are summarized in Table 7.

TABLE 7
Pharmacokinetic Parameters of Model Drug Used in Simulations

| | Dose^{a} | V/F | kₐ | kₑₗ |
|---|---|---|---|---|
| Drug | (mg) | (L) | (hr⁻¹) | (hr⁻¹) |
| d-Amphetamine | 20 (fed) | 247.0 | 0.744 | 0.067 |
| | 30 (fasted) | | | |

^{a}Dose represents mixed amphetamine salts dose. Twenty milligrams of the mixed amphetamine salts is equivalent to 12.5 mg of total amphetamine base and contain d-amphetamine and 1-amphetamine salts in the ratio of 3:1.

Parameters in the models, which represent GI transit effect, are gastric emptying rate constant and lag time of gastric emptying. The gastric emptying rate constant is zero order for the fed condition and first order for the fasted condition.

Variability of Model Parameters

In Monte Carlo simulations, variability of some or all model parameters is included in the simulations. Because effects of gastric emptying on the plasma concentration-time curve are being considered, variability of gastric emptying time and lag time of emptying was included in the simulations. Variability of other model parameters, on the other hand, was not included.

Gastric emptying time, lag time of emptying and their variability (standard deviation) were obtained from the literature. A lognormal distribution was chosen for all time parameters since time cannot be negative. T₅₀ is utilized for calculation of a first-order emptying rate constant in the fasted condition. Lag time of emptying in the fasted condition was selected based on phase 1, a period of motor inactivity, of MMC, which lasts approximately 30 to 60 min. Variability for lag time of emptying in the fasted condition was assumed to be 30 percent. Model parameters and their probability distribution used in the simulations are detailed in Table 8.

TABLE 8
Probability Distribution of Model Parameters
And Their Mean and Standard Deviation

| Parameters (unit) | Distribution | Mean ± SD (ref.) |
|---|---|---|
| Fed Condition | | |
| Lag time of emptying (hr) | Lognormal | 1 ± 0.37 (9) |
| Gastric emptying time (hr) | Lognormal | 5.7 ± 0.9 (5) |
| Fasted Condition | | |
| Lag time of emptying (hr) | Lognormal | 0.75 ± 0.22 (2) |
| T₅₀ (hr)^{a} | Lognormal | 0.5 ± 0.2 (6) |

^{a}First-order emptying rate constant is calculated from 0.693/T₅₀.

Determination of Amphetamine Absorption Profile from Mixed Immediate Release and Enteric-coated Pellets

Deconvolution of available plasma amphetamine concentration profiles (New drug approval package—Adderall XR clinical pharmacology and biopharmaceutics review (Approval date: October 2001). Website of CDER Freedom of Information Office, U.S. Food and Drug Administration. Retrieved Apr. 15, 2002, http://www.fda.gov/cder/foi/nda/2001/21303_Adderall_biopharmr.ddf) from commercial, mixed, immediate-release and enteric-coated pellets was performed using Kinetica 2000 software, version 3.0 (InnaPhase Corporation, Philadelphia, Pa.), to obtain the absorption profiles of d-amphetamines.

Average predicted peak concentrations data (Cₘₐₓ) from the simulations and observed Cₘₐₓ data in fed and fasted subjects are presented in Table 9. Simulated plasma concentration-time data curves are shown as mean plots. The average data values of 500 simulated plasma concentrations for each time point from 500 simulations were plotted in the mean plots. Vertical bars in the mean plots represent the standard deviation of 500 simulated concentrations for each time point.

TABLE 9
Summary of predicted Cₘₐₓ data and observed Cₘₐₓ data
of d-amphetamines

| Condition | Predicted Cₘₐₓ | Observed Cₘₐₓ | % |
|---|---|---|---|
| (Dose) | (ng/ml) | (ng/ml) | Difference^{a} |
| Fasted | 43.6 | 40.2 | 8.4 |
| (30 mg) | | | |
| Fed | 26.0 | 25.3 | 2.8 |
| (20 mg) | | | |

^{a}% Differences are presented as percentage of the observed Cₘₐₓ. The simulated data are quite accurate and close to the observed data.

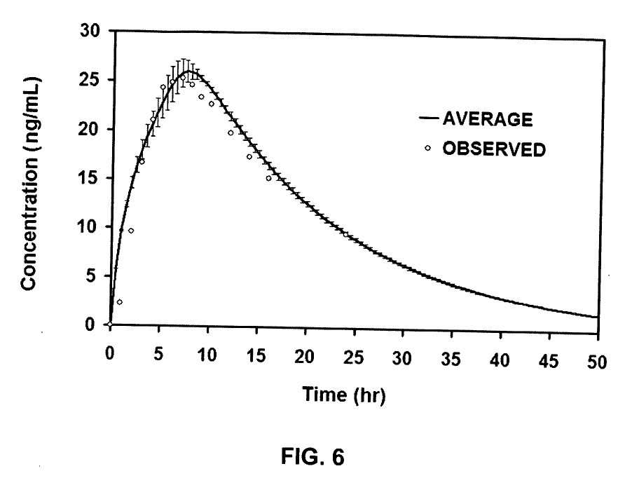

A mean plot of simulated plasma concentration-time curve of amphetamine from mixed immediate release and enteric-coated pellets in the fed condition are presented in FIG. 6. FIG. 6 shows that the simulated plasma concentration-time curve of amphetamine, after taking into account GI transit time and lag time of emptying in the fed condition, is very close to the reported amphetamine concentrations in plasma following oral administration of commercial mixed pellets in fed subjects. The predicted Cₘₐₓ differs from the observed value by only 2.8 percent (Table 9). Actual amphetamine concentrations are close to predicted lines. One explanation for the slightly lower concentration of the first two points is that there might be a delayed absorption of amphetamine from immediate-release pellets in the fed condition. In the presence of a meal, the immediate-release drug must dissolve and then find its way through the food to absorption surfaces of the stomach or travel into the intestine to be absorbed. Therefore, a short lag time of absorption of the drug from immediate-release pellets in the fed condition is not surprising.

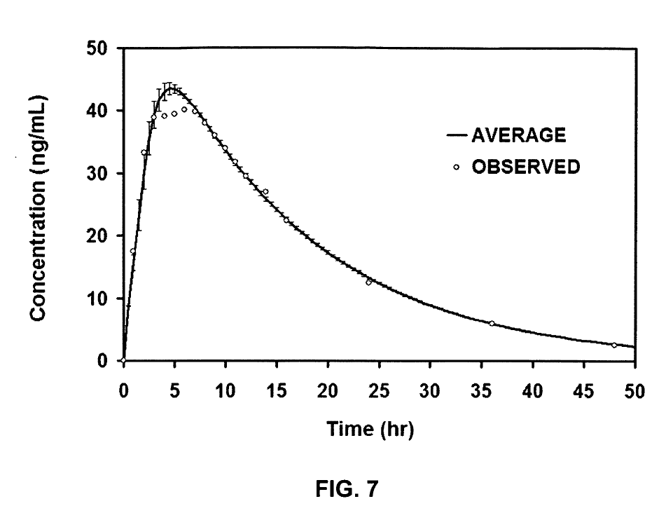

A mean plot of simulated plasma concentration-time data curve of amphetamine from mixed immediate release and enteric-coated pellets in the fasted condition is presented in FIG. 7. FIG. 7 shows that the simulated plasma concentration-time curve of amphetamine predicts quite well the reported amphetamine concentrations in plasma from commercial mixed pellets in fasted subjects. The predicted Cₘₐₓ differs from the observed value by only 8.4 percent (Table 9). The model slightly overestimates the peak concentrations of amphetamine between 4 and 6 hours.

Pharmacokinetic models used above incorporated the effect of gastric emptying on plasma concentration-time curve of amphetamine from mixed, immediate-release and enteric-coated pellets. The enteric-coated pellets did not release drug in gastric fluid. Data provided by the simulations give numerical and pharmacokinetic support that the plasma concentration-time curve data of amphetamine, when administered as mixed, immediate-release and enteric-coated pellets both in fed and fasted condition, do not produce a double-pulsed absorption pattern even though one-half of the drug is released quickly in gastric fluid and the other one-half of the drug is not released until two hours later in the in vitro dissolution test (FIG. 3). Unlike drug release in a dissolution chamber, in vivo drug release from enteric-coated pellets is influenced by both the GI transit and pH in the GI tract. Prolonged absorption of active ingredient from enteric-coated pellets is a result of GI transit characteristics under fed and fasted conditions, wherein the immediate-release drug is released quickly in the stomach and the enteric-coated drug is not released until the enteric-coated beads are “trickled” into the intestine.

The assumption of the model about instantaneous dissolution of enteric-coated pellets in the intestine seems to be valid, even though the in vitro dissolution profile of the commercial formulation showed that the amphetamine release took about 30 to 45 minutes in pH 6.0 medium. This small “sustaining” release in dissolution is insignificant for amphetamine when compared to the much larger variation of the gastric lag time and emptying. Using the assumption of instantaneous dissolution is advantageous in simplifying the model so that it can be applied to other mixed pellet formulations as long as the drug release time is relatively short. This example clearly shows that data generated by the simulations is quite accurate at predicting drug concentrations. In complex physiological systems like the human body, when considering the effects of product formulation, simulation data often vary by 100% or more from actual data, are preferred to be within 60% of actual data, more preferred to be within 40% of actual data, and most preferred to be within 30% of actual data.

### Example 4

This example shows experimental processes used to provide expected drug concentration versus time profiles for some drugs following administration of leaky enteric compositions. Pharmacokinetic modeling was applied for fed and fasted human subjects using known GI transit parameters to predict plasma drug concentrations following administration of new leaky enteric compositions. Monte Carlo simulation is applied to the models to include the effect of GI transit variability on simulated plasma concentrations of the drug from novel, leaky, enteric-coated pellets. Available pharmacokinetic data in the fed or fasted condition, depending on the drug, are compared to data generated from the simulation models.

Pharmacokinetic Models of Leaky Enteric-Coated Beads

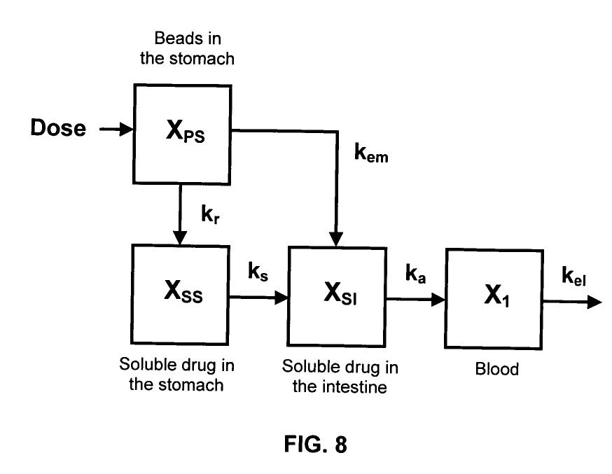

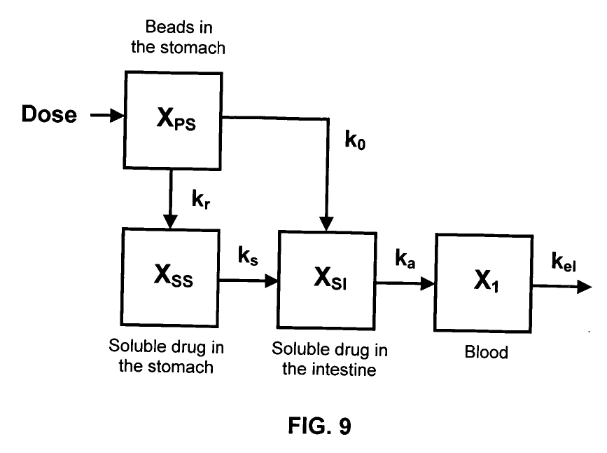

Compartmental diagrams illustrating pharmacokinetics of drugs from leaky enteric-coated beads in the fasted and fed condition are created and shown in FIGS. 8 and 9, respectively.

Pharmacokinetic model of leaky enteric-coated beads in fasted condition is presented in Equation 8.

$${C}_{1} = \frac{{k}_{a} D}{V} [ \frac{( {k}_{c} {k}_{s} - {k}_{em} {k}_{c} ) {ⅇ}^{- {k}_{c} t}}{( {k}_{a} - {k}_{c} ) ( {k}_{s} - {k}_{c} ) ( {k}_{el} - {k}_{c} )} + \frac{( {k}_{c} {k}_{s} - {k}_{em} {k}_{s} ) {ⅇ}^{- {k}_{s} t}}{( {k}_{a} - {k}_{s} ) ( {k}_{c} - {k}_{s} ) ( {k}_{el} - {k}_{s} )} + \frac{( {k}_{c} {k}_{s} - {k}_{em} {k}_{a} ) {ⅇ}^{- {k}_{a} t}}{( {k}_{s} - {k}_{a} ) ( {k}_{c} - {k}_{a} ) ( {k}_{el} - {k}_{a} )} + \frac{( {k}_{c} {k}_{s} - {k}_{em} {k}_{el} ) {ⅇ}^{- {k}_{el} t}}{( {k}_{a} - {k}_{el} ) ( {k}_{s} - {k}_{el} ) ( {k}_{c} - {k}_{el} )} ] \\ where {k}_{c} = {k}_{em} + {k}_{r} \qquad Eqn 9$$

For the fed condition, computer programming codes were developed using MATLAB computer language (The MathWorks, Inc., Natick, Mass.) to delineate the compartmental diagram.

Model Assumptions

Assumptions underlying pharmacokinetic models of leaky enteric-coated beads used in simulations are:

1) Drug pharmacokinetics are linear in the dosing range of interest. Thus, superposition for determination of plasma drug concentrations can be applied.

2) Leaky enteric-coated formulation is in multi-unit pellet/granule (multi-particulate) form.

3) Drug release from leaky, enteric-coated formulation in the stomach is adequately described by assuming a first-order process.

4) Upon transfer into the intestine, drug release from leaky, enteric-coated formulation in the intestine is instantaneous.

5) Once being released from the formulation into the intestine, the drug is absorbed by a first-order process.

6) Pharmacokinetics of the drug in the body are well described by a one-compartment open model.

7) Drug elimination from the body is a first-order process.

8) No assumptions are required to be exact, or even correct, so long as the model adequately approximates the processes described. Example 3 clearly validates that assumptions and models are adequate.

Model Parameters

Pharmacokinetic parameters of riboflavin-5-phosphate, ranitidine hydrochloride, and hydrochlorothiazide used in simulations were obtained by fitting of plasma concentration-time data from the literature (Zempleni, J.; Galloway, J. R.; McCormick, D. B. Pharmacokinetics of orally and intravenously administered riboflavin in healthy humans. American Journal of Clinical Nutrition 1996. 63, 54-66.; Abbreviated New Drug Application (ANDA) 074-467 Ranitidine hydrochloride Geneva Pharmaceuticals. Drugs@FDA Website, Retrieved from http://www.accessdata.fda.gov/scripts/cder/drugsatfda/.; Patel, R. B.; Patel, U. R.; Rogge, M. C.; Shah, V. P.; Prasad, V. K.; Selen, A.; Welling, P. G. Bioavailability of hydrochlorothiazide from tablets and suspensions. J. Pharm. Sci. 1984. 73 (3), 359-361.).

All data fittings were performed on data from immediate-release formulations using WinNonlin software, version 3.2 (Pharsight Corporation, Mountain View, Calif.). Table 10 summarizes pharmacokinetic parameters of all model drugs used in simulations.

TABLE 10
Pharmacokinetic Parameters of Model Drugs Used in Simulations

| | | | | |
|---|---|---|---|---|
| Dose | | | | |
| (mg) | F | V (L) | kₑₗ (hr⁻¹) | kᵣ (hr⁻¹) |

Riboflavin-5-phosphate

| | | | | | |
|---|---|---|---|---|---|
| 60 | 85% | 190.7 | 3.67 | 0.32 | 0.144, 0.347, 0.693 |

Ranitidine hydrochloride

| | | | | | |
|---|---|---|---|---|---|
| 300 | IR^{a} | 199.6^{b} | 0.641 | 0.239 | 0.144, 0.347, 0.693 |

Hydrochlorothiazide

| | | | | | |
|---|---|---|---|---|---|
| 100 | 100% | 105.9 | 0.94 | 0.13 | 0.144, 0.347, 0.693 |

^{a}Bioavailability of ranitidine from leaky enteric-coated beads was assumed to equal that of IR formulation.
^{b}This value represents V/F.

Bioavailability of 60 mg, immediate-release riboflavin was 36.4% (Zempleni,, et. al.). Bioavailability of 60 mg riboflavin from leaky, enteric-coated beads used in simulations was assumed to be 85% based on results shown in Example 1. Bioavailability of leaky, enteric-coated beads of ranitidine hydrochloride was assumed to be equal to that of immediate-release formulation. Bioavailability of 100 mg, immediate-release hydrochlorothiazide was 50.3% (Patel, et. al.). Bioavailability of 100 mg hydrochlorothiazide from leaky enteric-coated beads was assumed to be 100% in simulations.

GI transit parameters involved in simulations were gastric emptying of beads and gastric emptying of liquid. Gastric emptying of drug beads in fasted and fed condition are first-order and zero-order processes, respectively (Hardy, J. G.; Lamont, G. L.; Evans, D. F.; Haga, A. K.; Gamst, O. N. Evaluation of an enteric-coated naproxen pellet formulation. Aliment. Pharmacol. Ther. 1991. 5, 69-75.; Davis, S. S.; Khosla, R.; Wilson, C. G.; Washington, N. Gastrointestinal transit of a controlled-release pellet formulation of tiaprofenic acid and the effect of food. Int. J. Pharm. 1987. 35, 253-258.). Gastric emptying of liquid was a first-order process (Collins, P. J.; Horowitz, M.; Cook, D. J.; Harding, P. E.; Shearman, D. J. C. Gastric emptying in normal subjects—a reproducible technique using a single scintillation camera and computer system. Gut 1983. 24, 1117-1125) and was assumed to be a similar rate for both fasted and fed simulations. GI transit parameters used in simulations are shown in Table 11.

TABLE 11
GI Transit Parameters Used in Simulations

| | Gastric emptying time | |
|---|---|---|
| kₑₘ^{a} (ref.) | in fed condition (ref.) | t₅₀^{b} (ref.) |
| 1.39 hr⁻¹ (4) | 5.7 hr (5) | 0.25 hr (6) |

^{a}First-order gastric emptying rate constant of beads in fasted condition
^{b}Half-time for gastric emptying of liquid

Computer Simulations

All simulations were performed using MATLAB software, version 6.5 (The MathWorks, Inc., Natick, Mass.). The simulated plasma concentration-time curves of each model drug are visually compared to published literature data of immediate-release formulation.

### Example 5

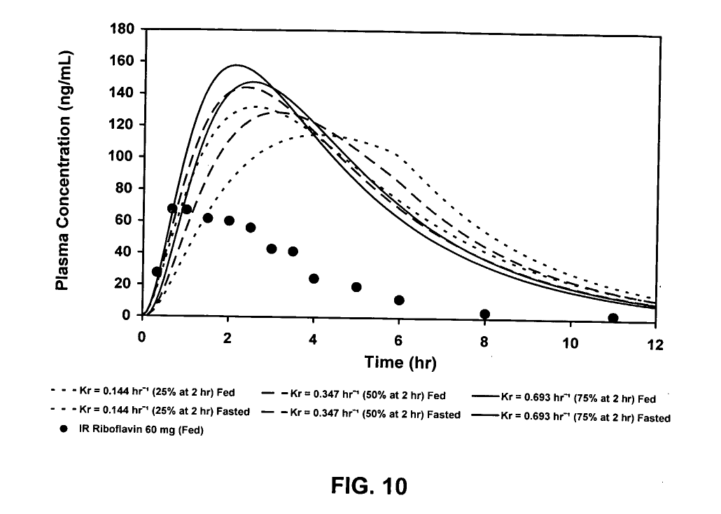

Example 1 establishes that the bioavailability of riboflavin is dramatically increased when the drug is released slowly in the stomach and trickles into the intestine. Bioavailability is decreased if the drug is trapped inside a composition and passes the absorption window before drug is released. As described in Example 4, FIG. 10 is a curve of drug concentration versus time for riboflavin. Pharmacokinetic data for riboflavin were obtained from Zempleni, et. al, Am J Clin Nutr. 1996; 63: 54-66. Data points from the immediate-release dosage form were obtained in fed subjects and bioavailability was 36.4% for the immediate-release dosage form and set at 85% from the new leaky enteric formulation, based on Example 1 above.

New enteric compositions can produce (see FIG. 10) an onset time to quantifiable plasma concentrations that is approximately equal to that from an immediate-release dosage form while also providing higher drug concentrations in the body and prolonged drug concentrations in the body. Each effect is dramatic and readily seen to be individually important and beneficial even if only one such effect occurs.

FIG. 10 is a curve of drug concentration in plasma versus time for riboflavin following administration to human subjects of IRF (immediate release formulation) active ingredient or as some novel leaky enteric compositions of riboflavin that give programmed release of their active ingredient into gastric fluid at first order rates as shown in the legend, which results in either 25%, 50%, or 75% release of their active ingredient contents over two hours in gastric fluid.

The release rates and first order character are given as only examples of the infinite number of release rates and types that can occur from drug dosage forms of the new invention. Any type or order of release rate or mixed release-rate from the new leaky enteric compositions is acceptable so long as the desired outcome is obtained. The data points for IR riboflavin are from Zempleni, et al., and solid lines are from data generated as described in Example 4. One of ordinary skill in the art will readily recognize that riboflavin is only one example of active agents that can benefit from the new invention. In fact, currently preferred, novel embodiments of formulations according to the present invention typically comprise active agents other than riboflavin.

A person of ordinary skill in the art will readily appreciate that when bioavailbility is increased and drug input occurs over a longer time period as compared to immediate-relaease formulations, as shown in examples herein, then dosing frequency can be reduced. Further, dosing frequency may be reduced even if bioavailability is not increased when drug input occurs over a longer time period as compared to immediate-relaease formulations. This is true even for drugs that do not have an absorption window. Note, for only one example, that aspirin and other non-steroidal inflammatory agents or other drugs that irritate stomach tissue are often enteric coated to protect the stomach from irritation by the active agent. Such irritation often is associated with undissolved particles of the agent that are exposed to gastric fluid following disintegration of immediate-release dosage forms of non-steroidal anti-inflammatory agents and other irritating drugs in gastric fluid. These drug particles contact the walls and tissue of the stomach and then drug that is dissolved in the diffusion layer surrounding the particles is in a high enough concentration to damage/irritate the stomach tissues. But, the enteric coating comes with a price in that there is a delayed release of the drug and a delayed onset of action that is not desirable for the patient who needs/desires faster relief.

The novel, leaky enteric compositions disclosed herein are well suited to deliver irritating drugs, including non-steroidal, anti-inflammatory agents because they entrap particles of the drug in the composition, thereby protecting the stomach tissues from damage/irritation, but also allowing a portion of the dose of active ingredient to dissolve into gastric fluid. This results in drug being released and available without the lag time associated with traditional enteric-coated (delayed release) dosage forms. In this case, the active ingredients may or may not have an absorption window but still benefit greatly from the instant invention even if the drug is well absorbed throughout the intestinal tract. And, the prolonged drug input into the body compared to immediate-release dosage forms when using the novel, leaky-enteric formulations also makes it possible to reduce dosing frequency as defined by the FDA. These and other active ingredients will be well recognized as even more preferable agents than riboflavin for use in the new leaky enteric compositions.

### Example 6

This example shows that a low solubility drug with an absorption window can be delivered into the upper intestine from the stomach in a more slowly controlled fashion than occurs with rapid, immediate drug release in the stomach. Not only a desirable pharmacokinetic outcome can be obtained but the pharmacodynamic effect of the drug also can be unexpectedly changed as shown in my European Patent Application PCT WO 03/015745 A1.

Hydrochlorothiazide was used as a model drug that has an “absorption window” in the upper intestine, and formulated into a gastric retention formulation (GRF) to be retained for a prolonged time in the stomach and provide slow, controlled release of drug in the stomach resulting in slow, controlled delivery of drug from the stomach into the intestine. Prior to conducting a bioavailability/bioequivalency study with the GRF, a drug dissolution profile was used to predict the expected in vivo absorption profile by convolution. Pharmacokinetic models were generated and validated using observed plasma concentration data from a reference and compared to a model provided in the literature. The best-fit model was selected to assess convolution to simulate plasma concentration profiles. Good correlation between predicted and observed pharmacokinetic outcome confirms reliability of experimental data simulated from mathematical model pharmacokinetic simulation experiments.

Two formulations for hydrochlorthiazide (an immediate release formulation (IR) and a gastric retention device (GRD) containing sustained release formulations (SR)) were administered in the bio-study (bioavailability study). A commercial tablet containing 50 mg of HCTZ was used as an IR control, and spray-coated beads equivalent to 50 mg of HCTZ were formulated for slow drug release (SR) and included in the GRF. Bioavailability and pharmacodynamics of HCTZ from a GRD were compared to those from an IR.

Monitoring concentrations of hydrochlorthiazide in the urine of healthy adult volunteers allowed comparison of the relative bioavailability of hydrochlorthiazide from the GRD formulation and from a conventional tablet. Participation involved at least two days for each treatment with at least 72-hours washout period between doses. An IR was given once and the GRD was repeated twice to test the reproducibility of the new dosage form. A 50-mg dose was chosen for the study because it was in the range of the recommended dose from the PDR (Physician's Desk References) and it produced concentrations high enough to make HPLC analysis efficient. Six subjects participated in the study, 4 healthy males and 2 healthy females. They were not allowed any food or drink containing caffeine, nor alcohol or other medications. Smokers and vegetarians were not included. Subjects fasted overnight and at least 2 hours following dosing. They voided their bladder before receiving a single dose of hydrochlorthiazide in each study and took the dose with 12 ounces of water. After dosing, subjects received a set of containers in which to collect their urine and a time sheet on which to record the time of urination. Subjects collected all urine within a 24-hour period after oral administration of the formulations. Urine samples were collected during the period 0-1, 1-2, 2-3, 3-4, 4-6, 6-8, 8-10, 10-12, 12-24, 24-36 and 36-48 hours. Urine samples were refrigerated until delivered to the researcher. The volume of urine collected was measured in order to calculate total amount of drug recovered. A modified method for HPLC (High performance Liquid Chromatography) assay of Papadoyannis et al., (Papadoyannis I N, Samanidou V F, Georga K A, Georgarakis E (1998) High pressure liquid chromatographic determination of hydrochlorothiazide (HCT) in pharmaceutical preparation and human serum after solid phase extraction, J. Liq. Chrom. & Rel. Technol., 21(11): 1671-1683.) was used to analyze small portions of urine samples for the drug content.

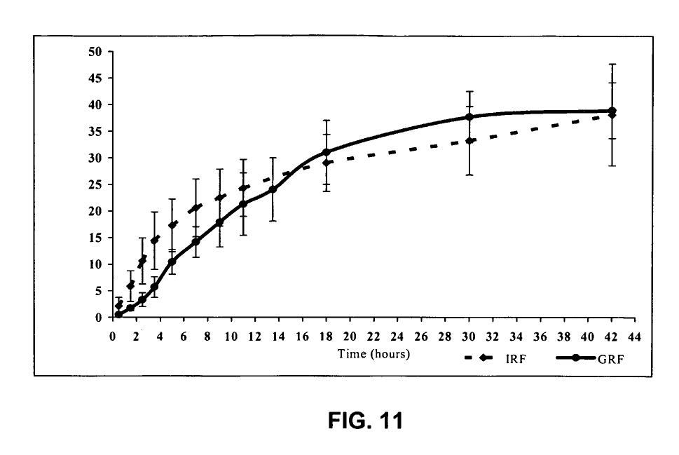

Pharmacokinetic parameters and urine output data following administration of either immediate release drug or slow drug input into the upper small intestine from a GRD containing hydrochlorothiazide were then obtained. Average pharmacokinetic parameters for each treatment under fasting conditions are provided in the following Table 12. FIG. 11 shows cumulative amount of drug excreted versus time. Elimination half-life (t_{1/2}) was approximately 7 hours. The values of A₀₋₃₆ were compared for statistical analysis because it was not possible to obtain the value at 48 hours for an IR from one subject due to the short half-life.

Mean A₀₋₃₆ₕ from IR (33.3mg, 66.6%) was found to be significantly different (P<0.05) relative to that from GRD (37 mg, 75.4%) in fasting conditions, although the difference is less than 10%. A difference of less than 20% is generally considered to be insignificant from FDA BA/BE guidance. From FIG. 11 [and Table 12, mean values for total drug absorbed and collected in the urine were equivalent, (A₀₋₄₈) were 38.12 mg (76.2%) and 38.95 mg (77.9%) for IR and GRD in fasting conditions, respectively. A₀₋₄₈ was based on assuming 50% of absorbed dose appears intact in the urine. Thus; the GRD resulted in essentially the same amount of drug being absorbed as from an IR up to 48 hours in fasting subjects. However, the effects on urinary excretion were surprisingly quite different.

TABLE 12
Mean pharmacokinetic parameters of hydrochlorothiazide from
6 subjects in fasting condition.

| | Treatments | |
|---|---|---|
| | IR | GRD |
| Total urinary recovery (0-48 h) (mg) | 38.1 ± 9.6 | 39.0 ± 5.2 |
| Maximum urinary excretion rate (mg/h) | 4.8 ± 1.7 | 2.5 ± 0.5 |
| Time for maximum urinary excretion rate (h) | 2.5 | 5 |
| Total average uring output (ml in 48 hours) | 6,068 | 7,467 |
| Cummulative average water intake in 48 | 6,013 | 7,380 |
| hours (ml) | | |

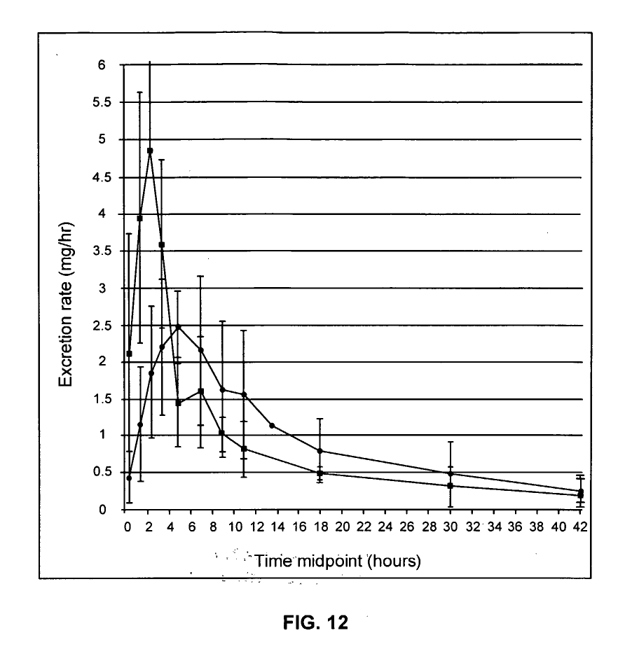

FIG. 12 shows that, as expected, a higher maximum excretion rate of drug (Rmax) occurred at an earlier time (tₘₐₓ) from the immediate release (IR) capsule than that from the new formulation (GRD) (4.84 mg/hr at 2.5 hr vs 2.5 mg/hr at 5 hr).

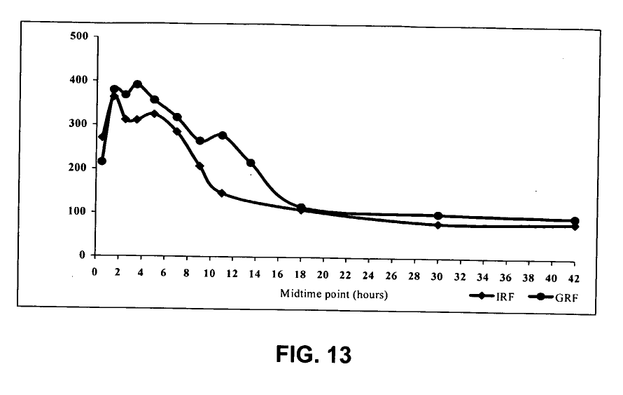

This example demonstrates that programmed release in gastric fluid give no change in F for hydrochlorthiazide (if the GRD stays in the stomach long enough) but drug efficacy is increased. Leaky enteric compositions also provide programmed release in gastric fluid and the same type of improved efficacy is expected. The rate of urine production was similar for both IR and GRD up to about 6-8 hours post-dosing (FIG. 13). This is quite unexpected since the initial amount of drug absorbed and drug concentrations in the body are less from the programmed drug release into gastric fluid compared to the commercial IR capsule. And, diuresis started decreasing for the IR capsule after 6-8 hours, whereas a relatively higher amount of diuresis was maintained for GRD for a longer time period.

The initial equal amount of diuresis from slow drug input into gastric fluid is surprising since less drug is absorbed initially from the GRD (Rmax 4.8 (μg/ml) at tₘₐₓ, 2.5 hours and 2.5 (μg/ml) at tₘₐₓ, 5 hours in fasting condition for IR and GRD, respectively) which now teaches that less drug input can be equally effective, which is not common for drugs. In fact, if less amount of drug is input, less effect is expected but the opposite effect occurred with this new GRD and the diuretic.

Drug effect on urine production from the GRD continued until approximately 15 hours (see FIG. 13).

Table 12 shows that increasing body fluid excretion in healthy, normal subjects stimulated water-intake. Total amount of urine production was higher from the same dose in a GRD compared to IR, which can be attributed to prolonged drug input from GRD followed by a feedback increased amount of water-intake to compensate for the unexpected increased drug effect.

This overall increased effect is also surprising (in addition to the initial greater effect with a smaller drug input discussed above) since it is well known that in order to increase diuretic effect it is necessary to increase the drug dose. In fact, most drug response curves are log-linear, which means that usually an increase in effect is less (smaller percentage) than the increase in dose after an initial response threshold is crossed. But, in this case, the bioavailability of drug under fasting conditions was essentially equal, but the diuretic effect was increased 27% as shown in Table 12 above.

Results from this bioavailability study of hydrochlorthiazide establishes not only that the device was retained long enough to release all or most drug in the stomach, but also that the dosage form provided drug release into gastric fluid that resulted in slow drug input into the absorption window area to prolong drug effect. The desirable outcomes occur because the dosage form allows the drug to be released in gastric fluid in a manner that provides slow and prolonged, or “trickle,” drug input into the upper small intestine. This dosage form can improve patient care by, amongst other things, (1) avoiding high drug peak concentrations that may induce undesirable side effects (see side effects information below), (2) increasing drug effect per dose administered and/or (3) achieving prolonged drug effect.

Side effects reported from the drug hydrochlorothiazide administered to human subjects as outlined above were:
Three out of 7 subjects reported side effects from an IR dosage form between 4-6 hours post-dosing.
Adverse reactions reported were severe or moderate headache, dehydration, and fatigue.
One subject did not continue in the study due to severe headache, dehydration, and fatigue.
No adverse reactions were reported from the same dose of hydrochlorthiazide when drug released in gastric fluid as described above.

This example shows that slow drug input, which is produced following administration of leaky, enteric-coated dosage forms as described elsewhere herein, is unexpectedly beneficial in increasing drug effect, particularly for therapeutics having an absorption window. Greater advantages are expected for drugs that have a lower bioavailability from immediate release dosage forms due to regional absorption limitations, e.g., as shown for riboflavin. Disclosed embodiments of the present invention effectively decrease drug peak concentrations by 20% or more, decrease drug side effects by 10% or more, prolong drug concentrations in the body sufficiently to allow a decrease in frequency of dosing, increase drug bioavailability by 10% or more, and improve patient compliance relative to known, immediate-release formulation technology. Not all of these beneficial effects occur for every drug but each effect may occur depending on the active agent involved, and only one such beneficial effect is sufficient reason to use the new compositions disclosed herein.

Prolonging drug concentrations for drugs with an absorption window using multiparticulate bead or granule formulation, capsules, or tablets other than floating (relatively non-effective) dosage forms or gastroretentive devices, while maintaining acceptable bioavailability has been considered impossible because known, sustained-release formulations pass the site of absorption before all the drug is released. The novel, leaky enteric compositions disclosed herein release drug slowly while in the stomach and then release all or most of the remainder of the drug in the upper small intestine before the composition passes the absorption window. This allows prolonged drug absorption and is beneficial even if bioavailability is reduced.

### Example 7

Hydrochlorthiazide, which is known to have an absorption window in the intestinal tract and has limited absorption related to limitations on dissolution (Dressman J B, Fleisher D, Amidon G L., Physicochemical model for dose-dependent drug absorption, J Pharm Sci 1984; 73(9): 1274-9.) was prepared as a leaky enteric composition according to the present invention. Such dosage form was prepared by spray-layering drug on nonpareil sugar beads and then applying an enteric coating formulated to allow drug to be released in gastric fluid at programmed rates. Enteric coating typically would prevent drug release in gastric fluid. But hydroxyproply methylcellulose (HPMC) was used in this example, which allowed drug leakage into gastric fluid and then provided rapid release of remaining drug from the formulation when exposed to intestinal fluid

The formulations were prepared and applied on sugar beads as outlined in Example 2.

TABLE 13
Leaky Enteric-Coated Beads Formulations of Hydrochlorothiazide

| | | Amount of Leaky |
|---|---|---|
| | Composition of Leaky | Enteric-Coating Polymer |
| Formulations | Enteric-Coating Polymer | (% of Drug-Loaded Beads)^{c} |
| HCTZ1 | EUD^{a} with 20% HPMC^{b} | 7.5% |
| HCTZ2 | EUD with 5% HPMC | 5% |
| HCTZ3 | EUD with 5% HPMC | 7.5% |
| HCTZ4 | EUD with 5% HPMC | 10% |

^{a}Eudragit ® L30D-55 (EUD). Working titles of coating formulation. Final coating formulation given below.
^{b}Hydroxypropyl methylcellulose (HPMC)
^{c}Weight gain of beads coated with the coating material after drying the coated beads.

TABLE 14
Compositions of Leaky Enteric-Coating of Hydrochlorothiazide
Formulations

| | |
|---|---|
| Ingredients | Formulations (% |
| (Solid | of Total Coating Materials) |

| Composition) | HCTZ1 | HCTZ 2 | HCTZ 3 | HCTZ 4 |
|---|---|---|---|---|
| Eudragit ® | 58.8 | 64.5 | 64.5 | 64.5 |
| L30D-55 | | | | |
| Talcum | 29.4 | 32.3 | 32.3 | 32.3 |
| HPMC E5 | 11.8 | 3.2 | 3.2 | 3.2 |

TABLE 15
Hydrochlorothiazide loading solution

| Ingredient | Amount | |
|---|---|---|
| Hydrochlorothiazide | 5.0 | g |
| PVP K-30 | 3.0 | g |
| Deionized water | 30.0 | ml |
| 95% Ethanol | 500.0 | ml |

Accurately weighed hydrochlorothiazide was dissolved in 500 ml of ethanol (solution may be warmed to facilitate the dissolution). PVP K-30 was dispersed in 30 ml of deionized water before being added to hydrochlorothiazide solution and well mixed.

TABLE 16
Eudragit ® L30D-55 with 5% HPMC dispersion

| Ingredient | Amount |
|---|---|
| Eudragit ® L30D-55 | 33.3 g |
| Triethyl citrate | 1.0 g |
| Talcum | 5.0 g |
| HPMC E5 | 0.5^{a} |
| Deionized water | 115.0 |

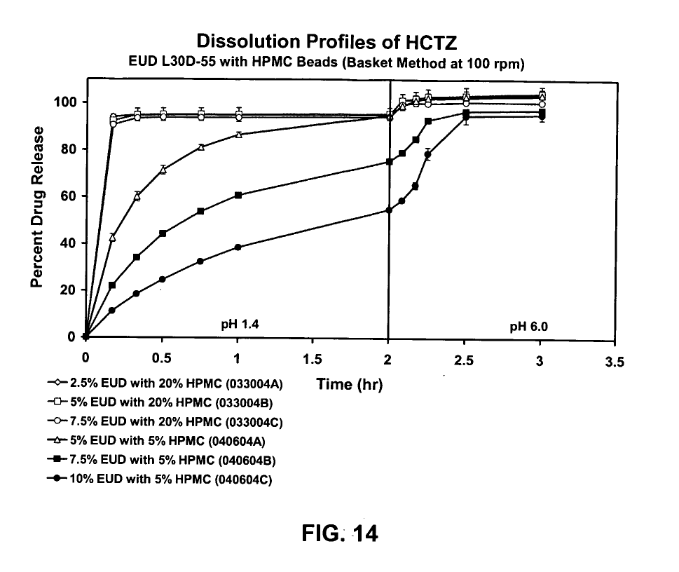

Accurately weighed HPMC E5 was dispersed in approximately 15 ml of hot deionized water. Fifteen (15) ml of cool deionized water were added to the well-dispersed HPMC and the solution was stirred until clear. Talcum was dispersed in the remaining deionized water. Talcum dispersion was added to HPMC solution and kept stirring. Eudragit® L30D-55 was accurately weighed into a beaker. Triethyl citrate was added to Eudragit® suspension and gently mixed. The HPMC and talcum dispersion was then added into Eudragit® mixture and gently mixed. This mixture was kept gently stirring. The amount of HPMC used in studied formulations was calculated as a percentage of Eudragit® polymer solid (Eudragit® polymer suspension contains 30% polymer solids.) Dissolution results are shown in the following FIG. 14, which provides percent drug release over time for hydrochlorothiazide as a leaky, enteric coated dosage formulation.

FIG. 14 clearly shows the leaky enteric composition released therapeutic agent into gastric fluid at a programmed rate depending on the new composition used, and then rapidly released the remainder of the therapeutic agent after transfer into intestinal fluid. It is generally preferred that at least 10%, and in more preferred embodiments that at least 20% therapeutic agent, is released in gastric fluid. It is generally preferred that 75% or more therapeutic agent not released in the gastric fluid be released in one hour or less following transfer into intestinal fluid for therapeutic agents with a window of absorption in the duodenum. It is a more preferred embodiment that 75% or more of therapeutic agent not released in the gastric fluid be released in one-half hour or less following transfer into intestinal fluid for therapeutic agents with a window of absorption in the duodenum.

### Example 8

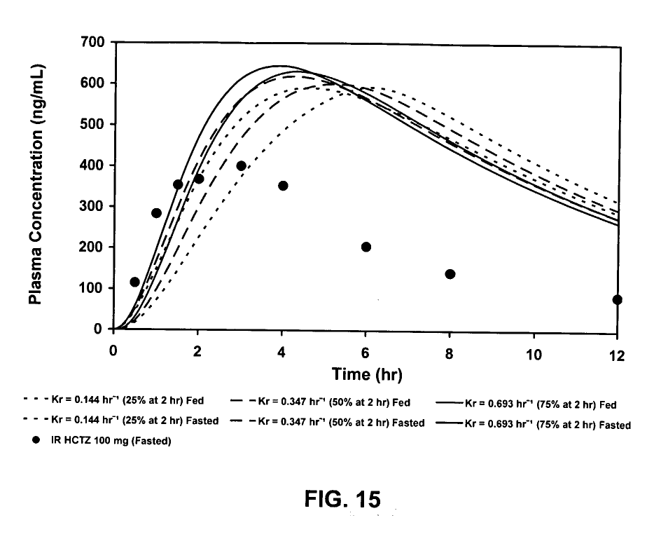

This Example 8, in combination with Example 6, provides pharmacokinetic effects on a drug with an absorption window in the upper portion of the intestine when formulated as a leaky enteric formulation with programmed drug release in gastric fluid compared to an immediate release dosage form of the drug. For data points and lines in FIG. 15, pharmacokinetic data for hydrochlorthiazide were obtained from Patel, et. al, J. Pharm. Sci., Bioavailability of hydrochlorothiazide from tablets and suspensions, 1984, 73(3),359-361. Data points from the immediate-release dosage form reflect administration to fasted subjects and bioavailability set at 50.3% for the immediate-release dosage form and set at 100% from the new, leaky enteric formulation. Drug formulated into a new, leaky enteric composition is delivered more slowly and in a more prolonged manner into the intestine than occurs from an immediate release dosage form as taught herein. Bioavailability may not increase as much as shown in FIG. 15 for hydrochlorthiazide or other drugs, but the resulting change in drug concentration profile in the body is beneficial as taught previously. Increased bioavailability from the new enteric composition is an additional benefit when it occurs. Since data points in FIG. 15 represent fasting subjects receiving an immediate-release dosage form of hydrochlothiazide it is expected that the bioavailability will be increased in the presence of food and also may be increased from the leaky enteric composition that provides programmed initial drug release in the stomach and then rapid release when transferred into the upper small intestine. Data in FIG. 15 allow presentation of the maximum possible increase in bioavailability for hydrochlorthiazide.

Parameters for stomach transit times in both the fed and fasted conditions are presented in Example 3. Drug release patterns from the new, leaky enteric formulation are approximated as first-order release profiles with k vales of 0.144 hr⁻¹, 0.347 hr⁻¹, or 0.693 hr⁻¹ as shown in FIG. 15.

These data for hydrochlorthiazide in FIG. 15 are generated as described in Example 4. While the drug was released in a first-order fashion from the new compositions as described earlier and shown by the rate constants in FIG. 15, the present invention anticipates all types of drug-release mechanisims that provide programmed release in gastric fluid. It can be seen, for example, that a first order release mechanism releases 25% of the active ingredient in gastric fluid in two hours when the rate constant for release is 0.144 hr⁻¹ Likewise, drug release in gastric fluid is either 50% or 75% in two hours when the first-order release rate of active ingredient in gastric fluid is 0.347⁻¹ or 0.693⁻¹, respectively. It generally is preferred that the leaky enteric compositions release between 10% or more and 90% of the active ingredient in gastric fluid in two hours, independent of release mechanism, and more preferred that the leaky enteric compositions release between 20% or more and 90% of the active ingredient in gastric fluid in two hours, independent of release mechanism, and even more preferred that the leaky enteric compositions release between 10% or more and 35% of the active ingredient in gastric fluid in two hours, independent of release mechanism.

FIG. 15 shows higher drug concentrations in plasma for this drug with an absorption window as expected due to higher bioavailbility, and very substantial extension of drug concentrations in the body versus time compared to the known formulation of hydrochlorthiazide, even in fasted patients. Drug concentrations versus time are even more extended in fed patients. Leaky enteric composition data do not show the usually undesirable but required delay in drug absorption known to occur with traditional enteric coatings, which leads enteric compositions known prior to the present invention to be classified as delayed-release drug products by the FDA. Even if bioavailability is not increased, the leaky enteric composition still releases drug in gastric fluid, thus necessarily avoiding the type of delay that occurs when drug is not released until the composition has completely left the stomach and gastric fluid. Generally, adding a portion of immediate-release drug formulation to the enteric composition is not required since the leaky enteric release formulation already quickly provides drug concentrations in the body. But, if desired, the enteric composition can also comprise some immediate release drug. For example, in the beads of this example some immediate release drug can be over-coated or spray-layered on top of the enteric composition for even more rapid dissolution or burst effect of some drug, or included in uncoated form. In some cases, the drug may be incorporated directly into the enteric coating material, which would allow more rapid release of some drug. This would not, of course, satisfy the usual requirements for an enteric composition in that the drug incorporated into the coat now will be released into gastric fluid rather than being protected from gastric fluid, thereby meeting a key objective of the current invention.

This example 8, in combination with other examples, illustrates a novel enteric composition formulation with unexpected properties for a drug with a window of absorption that results in sustained drug input into the body without substantial delay or lag time for drug absorption, and increased drug efficacy for the same drug dose compared to traditional, immediate-release formulations of the drug. Example 6 illustrates that there are substantial, improved pharmacodynamics and reduced side effect benefits that result from providing more prolonged input of this absorption window drug using the disclosed formulations of the present invention. These effects occur even if bioavailability from the new dosage form is equivalent to bioavailability from the IR formulation. The combination of desirable effects was previously thought mutually exclusive given the known physiological absorption window and drug characteristics. The unique outcomes are made possible by the embodiments of the new composition disclosed herein that provides drug input in a programmed fashion due to programmed drug release in gastric fluid while the formulation is retained in the stomach, followed by rapid release of any remaining drug in the formulation when the formulation enters the intestine.

### Example 9

Ranititdine is well absorbed if introduced as an immediate-release formulation into the stomach or into the upper small intestine, but is poorly absorbed if introduced into the colon. Advantages and difficulties of formulating ranititdine as a traditional, sustained-release formulation are discussed in U.S. Pat. No. 5,407,687.

The general process for making and preparing the enteric composition of ranitidine that releases drug in gastric fluid was according to Example 2. Specifics for ranitidine are given below.

TABLE 17
Leaky Enteric-Coated Beads Formulations of Ranitidine Hydrochloride

| | | Amount of Leaky |
|---|---|---|
| | Composition of Leaky | Enteric-Coating Polymer |
| Formulations | Enteric-Coating Polymer | (% of Drug-Loaded Beads)^{b} |
| RTD1 | EUD^{a} with 33% lactose | 7.5% |
| RTD2 | EUD with 33% lactose | 10% |
| RTD3 | EUD with 33% lactose | 12.5% |
| RTD4 | EUD with 33% lactose | 15% |
| RTD5 | EUD with 50% lactose | 10% |
| RTD6 | EUD with 50% lactose | 12.5% |
| RTD7 | EUD with 50% lactose | 15% |

^{a}Eudragit ® L30D-55 (EUD)
^{b}Amount of leaky enteric-coating polymer is presented as an amount of Eudragit ® L30D-55 polymer solid (in leaky enteric-coat layer) coated onto drug-loaded beads. Eudragit ® L30D-55 suspension contains 30% polymer solid.

TABLE 18
Compositions of Leaky, Enteric-Coated Layer
of Ranitidine Hydrochloride Formulations

| Ingredients | | | | | | | |
|---|---|---|---|---|---|---|---|
| (Solid | Formulations (% of Total Coating Materials) | | | | | | |
| Composition) | RTD1 | RTD2 | RTD3 | RTD4 | RTD5 | RTD6 | RTD7 |
| Eudragit ® L30D-55 | 54.5 | 54.5 | 54.5 | 54.5 | 50.0 | 50.0 | 50.0 |
| Talcum | 27.3 | 27.3 | 27.3 | 27.3 | 25.0 | 25.0 | 25.0 |
| Lactose | 18.2 | 18.2 | 18.2 | 18.2 | 25.0 | 25.0 | 25.0 |

TABLE 19
Ranitidine Loading Solution

| Ingredient | Amount | |
|---|---|---|
| Ranitidine hydrochloride^{a} | 7.5 | g |
| PVP K-30 | 2.0 | g |
| Hydroxypropyl cellulose | 1.0 | g |
| (HPC) EXF | | |
| Deionized water | 60.0 | ml |

Accurately weighed HPC EXF was dispersed in 30 ml of hot deionized water. Cool deionized water was added to the well-dispersed HPC and the solution was stirred until clear. PVP K-30 was then added and well mixed. Finally, ranitidine was added to the solution and stirred until dissolved.

TABLE 20
Eudragit ® L30D-55 with 33% lactose dispersion

| | | |
|---|---|---|
| Eudragit ® L30D-55 | 50.0 | g |
| Triethyl citrate | 1.5 | g |
| Talcum | 7.5 | g |
| Lactose | 5.0^{a} | g |
| Deionized water | 100.0^{b} | ml |

Accurately weighed lactose was dissolved in 50 ml of deionized water (solution may be warmed to facilitate the dissolution). Talcum was dispersed in the remaining deionized water. Talcum dispersion was added to lactose solution and kept stirring. Eudragit® L30D-55 was accurately weighed into a beaker. Triethyl citrate was added to Eudragit® suspension and gently mixed. The lactose and talcum dispersion was then added into Eudragit® mixture and gently mixed with continuous stirring. The amount of lactose used in studied formulations was calculated as percentage of Eudragit® polymer solid (Eudragit® polymer suspension contains 30% polymer solid). The volume of deionized water varied as needed to sufficiently dissolve lactose (generally, one part of lactose can be comfortably dissolved in 10 part of water).

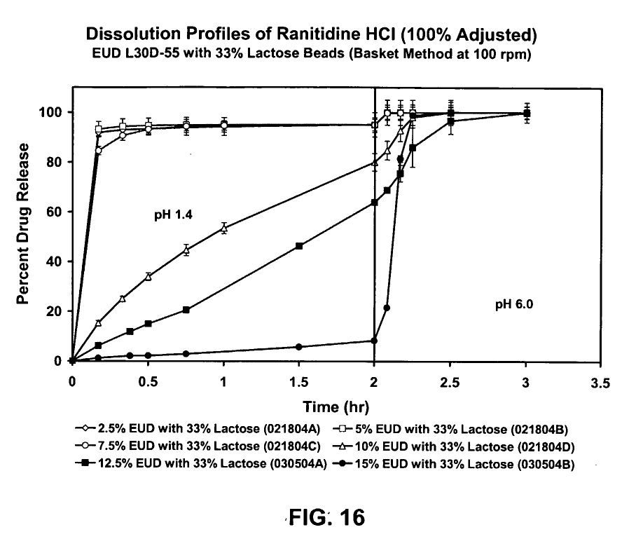

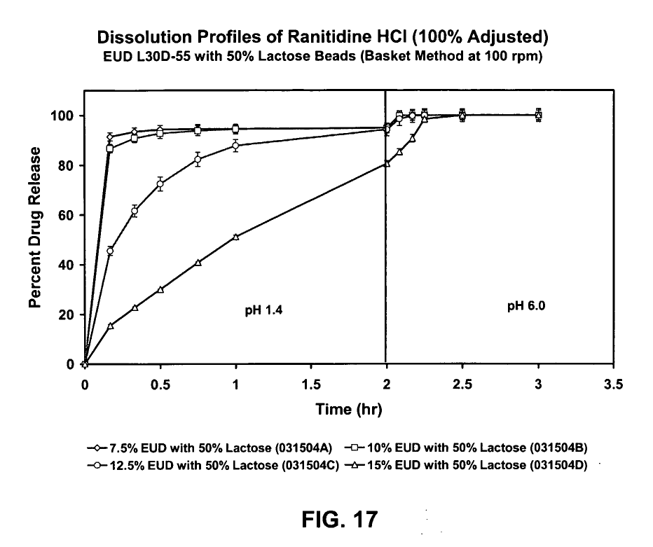

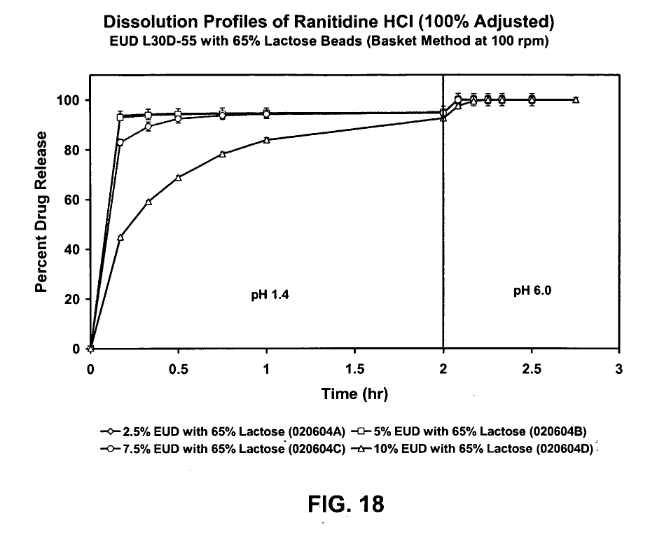

FIGS. 16-18 illustrate drug release from some embodiments of the present composition in dissolution testing. FIGS. 16-18 in combination with other figures herein show that formulation of the presently disclosed, novel, leaky enteric compositions can be easily controlled by one of ordinary skill in the art to obtain a variety of useful drug dissolution patterns in gastric fluid ranging from zero-order through mixed-order and first-order dissolution kinetics. Then, drug remaining in the composition when it leaves the gastric fluid is quickly released after exposure of the composition to intestinal fluid. In many cases this release from the composition after exposure to intestinal fluid is more rapid than occurs with known, effective enteric compositions. This likely is because the new compositions are “weakened” by the inherent structure that results from the combination of the composition formulation and exposure to gastric fluid.

### Example 10

This example shows prolonged drug concentrations using embodiments of the novel drug dosage formulations disclosed herein with equivalent bioavailability to ranitidine, another drug with an absorption window. IR data points are in fasted patients from the FDA website (Drugs@FDA, ANDA#074-467 Geneva Pharmaceuticals) for the drug product Zantac (ranitidine).

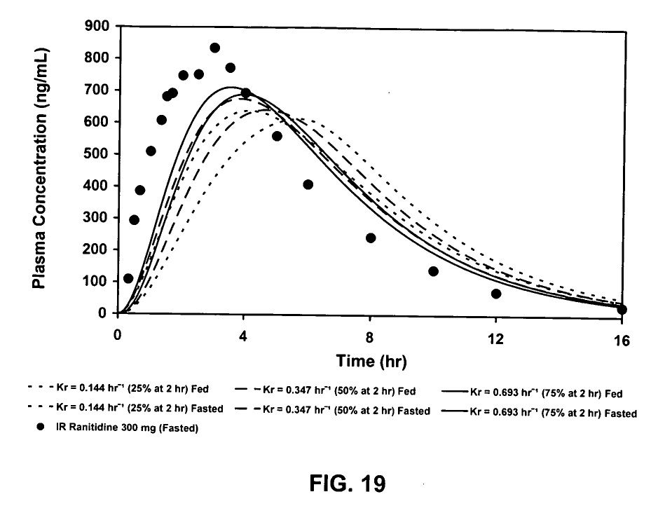

These data for ranitidine in FIG. 19 are generated as described in Example 4. While the drug was released in a first-order fashion from the new compositions as described earlier and shown by the rate constants in the FIG. 15 footnote, this invention anticipates all types of drug release mechanisims that provide programmed release in gastric fluid. It can be seen, for example, that a first-order release mechanism releases 25% of the active ingredient in gastric fluid in two hours when the rate constant for release is 0.144 hr⁻¹ Likewise, the drug release in gastric fluid is either 50% or 75% in two hours when the first-order release rate of active ingredient in gastric fluid is 0.347 hr⁻¹ or 0.693 hr⁻¹, respectively.

Data from FIG. 19 are for equal bioavailability from all formulations. It is known that average stomach emptying rates vary from study to study and individual to individual and have been reported to vary from at least as much as kₑₘ=1.4 hr⁻¹ to kₑₘ=0.5 hr⁻¹. Table 11 shows that kₑₘ was assumed to be 1.38 hr⁻¹ for other drug concentration vs. time data for fasting subjects but for the data in FIG. 19, kₑₘ=0.5 hr⁻¹ was used. FIG. 19 shows slower and more prolonged drug input from leaky enteric compositions with more sustained drug concentrations in the body than from an immediate-release formulation for ranitidine in both fasted (when kₑₘ=0.5 hr⁻¹) and fed states. When kₑₘ=1.4 hr⁻¹ in the fasted state for the leaky enteric coated compositions of FIG. 19, then the drug concentrations vs. time curves are almost identical for all three Kr values, and are quite close to the curve for the IR dosage form. Thus, it can be seen that for some individuals, depending on stomach emptying rate, the new leaky enteric compositions will be more beneficial than for others. It is also anticipated that additional ingredients that decrease kₑₘ (such as oils or fats or drugs as non-limiting examples) will be useful ingredients in the leaky enteric compositions. Further, some drugs with an absorption window also undergo first-pass metabolism as do some non-absorption window drugs. Thus, providing a slower input from the new leaky enteric compositions may result in either increased bioavailability, decreased bioavailability, or no change in bioavailability depending on what effects dominate for the specific drug. Even in the case of decreased bioavailability the leaky enteric compositions are beneficial because of other changes in the drug concentration versus time delivery profile, such as decreased maximum drug concentration in the plasma or sustaining drug input over time when compared to immediate release compositions.

Drug peak concentrations, as expected and generally desirable, are lower when drug input is slower and more sustained, compared to more rapid and less sustained drug input. U.S. Pat. No. 5,407,687 teaches the need for, but the difficulties associated with preparing, a sustained-release formulation to produce more prolonged drug input compositions with more sustained drug concentrations in the body than from an immediate-release formulation for ranitidine.

The solution provided by U.S. Pat. No. 5,407,687 is to produce a laminated, bi-layer tablet containing the drug in a fixed ratio of drug between one immediate-release drug layer and a second, sustained-release drug layer. The fixed ratio, in view of the peculiar properties of ranitidine, is required to obtain sustained drug concentrations in the body (not obtained by known systems).
That is, these (known) systems do not allow for the balance that must be made between the amount of drug immediately released and the amount of time over which the remaining drug in the sustained release (SR) portion is released. For example, if too much ranitidine is present in the immediate release (IR) portion, the result is essentially the same as that obtained with the commercially available tablets, i.e. an immediate release formulation. Conversely, if too little ranitidine is present in the immediate release portion, the resulting formulation exhibits poor bioavailability.

These “peculiar properties” result because ranitidine has site specific absorption, and is known to produce a double-peak in some concentration versus time curves. The double peak is not readily apparent in the fasting data in human subjects shown above and is not present in the new formulations data that did not include the possibility of multiple windows of absorption, absorption at different rates from different sites, or biliary recycling, all of which have been proposed and challenged in the literature.

While the equipment to make bi-layer tablets is well known in the art the process is not without substantial problems. Tablets often delaminate and production times and complexities are extended due to the multiple steps and multiple formulations involved. These problems are avoided by disclosed embodiments of the present invention, which has the distinct added advantage of not requiring multiple formulations or combinations in fixed amounts of IR and SR drug. While IR drug can be added to embodiments of the compositions of the present invention, this is not required because the leaky enteric composition can be formulated to either begin drug release immediately or after a short lag-time following consumption by a patient. And, the problem of poor bioavailability that occurs in SR formulations that entrap drug too long is avoided because drug is rapidly released upon entering the intestine with disclosed embodiments of the compositions of the present invention. For some drugs like ranitidine, that are well absorbed when introduced either into the duodenum or jejunum, release from the leaky enteric composition is formulated to provide programmed release in gastric fluid followed by a release pattern that is sustained over two to four hours, or even longer, in intestinal fluid, much like the sustained release of bilayer tablet of U.S. Pat. No. 5,407,687 provides sustained release in intestinal fluid. Or, embodiments of the leaky, enteric-coated drug formulations may be combined with sustained-release formulations that provide drug over a release period of up to 8 hours in intestinal fluid. Distinct advantages of the instant invention are that both bilayer tablets and fixed ratio of IR drug to sustained input amounts of drug can be avoided.

Applying superposition principle to data in FIG. 19 illustrates that if the dose is increased, such as by doubling, for one example, the differences in drug concentration peak values and values at prolonged times, such as 6, 8, 10, or 12 hours, for example, between the IR formulation and the new compositions will increase. This is because drug absorption is not sustained from the IR composition but is sustained from the new compositions. Thus, the frequency of dosing can be reduced, which is highly desirable but not known with traditional enteric compositions.

In one preferred embodiment, leaky enteric compositions are provided as compressed tablets. In this case particulates, such as beads or granules, are coated with enteric composition that may or may not release drug in gastric fluid if administered without compaction, but which do release drug in gastric fluid when administered as compacted tablets. This is because the compaction forces can convert non-leaky enteric compositions into leaky enteric compositions. The enteric composition particles may be mixed with usual tabletting excipients for compaction, or in a more preferred embodiment the enteric composition particles are coated or “layered” with tabletting excipients that are beneficial in promoting tablet disintegration in gastric fluid and tablet compaction. Other known excipients also are anticipated, such as lubricants, colors, flavors, surfactants, and all other types of appropriate excipients for making pharmaceutical formulations. These excipients and their uses are well known in the art.

Traditional, enteric-coated particulates, such as beads, granules or powders, which do not release drug in gastric fluid can be treated by chemical, physical, or mechanical methods to convert the composition into a leaky enteric composition. A few examples of treatments possible includes use of solvents, such as porogenic solvents that provide fluid ingress pores upon removal from the formulation, thermal methods, including heat-freeze cycle(s), granulation equipment, and application of pressure, such as with roller and other mills or tablet machines. Compositions that do release drug in gastric fluid can be filled into capsules for oral administration or compacted into tablets. Chewable tables are anticipated wherein mechanical forces that convert some or all of a non-leaky enteric composition into a leaky enteric composition includes the chewing process.

### Example 11

U.S. Pat. No. 6,399,086 teaches that β-lactam antibiotics have a specific absorption site in the small intestine. The '086 patent also teaches that there is a need for a dosage form that provides a burst effect by releasing about 50% of the drug within 3 to 4 hours of administration, and release of the remainder of the drug at a controlled rate. Such dosage form may comprise a β-lactamase inhibitor. AUC or bioavailability of the β-lactam antibiotic was significantly lower for the sustained release formulation, probably because the drug was trapped in the sustained-release matrix when passing the absorption window. In fact, U.S. Pat. No. 6,399,086 states that:
In principle, extending the residence time of the antibiotic drug in the GI tract by the sustained release formulation may increase, in theory, the GI adverse effects associated with amoxicillin therapy. Obviously, such phenomenon of increased risk of side effects is a limitation for the development of amoxicillin controlled-release formulations. However, in the formulations of the present invention, the unabsorbed portion of the dose that had a prolonged transit time in the GI tract is captured within the matrix formulation and is not available to interact with the intestine epithelia and/or flora, thus eliminating the danger of exposing the patient treated with the formulations of the present invention to said adverse side effects.

Thus, the drug is likely to cause less undesirable gastrointestinal side effects because it is trapped inside the dosage form and not available. And, if the drug is released after passing the absorption window then it is not only not available for the patient but it is then available to be released lower in the intestinal tract to cause undesirable gastrointestinal side effects. This decrease in bioavailability and potential increase in side effects is highly undesirable.

PCT WO 94/27557 discloses thermal infusion wax matrix formulations of amoxicillin and clavulanate, reports to provide prolonged release of both compounds, and teaches the difficulty and need of formulation techniques to provide prolonged input for both drugs. Only 19% of the amoxicillin is released in 6 hours from the prolonged release formulation. Thus, bioavailability is expected to be very low, as reported in U.S. Pat. No. 6,399,086, since the drug will be entrapped in the wax matrix when passing the absorption window. Although the clavulanate is released faster, it too can be trapped in the wax matrix and pass the absorption window in those cases when the tablet is taken on an empty stomach only a short time before arrival of the house-keeper wave.

Thus there remains a need for a composition that will provide sustained input of β-lactam antibiotics without entrapping the drug such that the bioavailability is significantly reduced, and without increasing the potential for making gastrointestinal side effects worse than occurs with immediate-release dosage forms of these drugs. An increase in bioavailability relative to immediate release dosage forms is not required to obtain useful benefits in patient care. A change in drug input pattern that extends useful drug concentrations in the body relative to immediate-release dosage forms can reduce dosing frequency, and can improve patient care even if frequency of dosing is not reduced. New leaky enteric compositions disclosed herein are particularly useful for the types of therapeutic agents disclosed in U.S. Pat. No. 6,399,086 and PCT WO 94/27557.

For one example, leaky enteric compositions are particularly suited to deliver combinations of β-lactam antibiotics or other combinations of drugs where the effect is synergistic and influenced by the pharmacodynamic/pharmacokinetic effects of one or both drugs. Using amoxicillin and clavulanic acid as examples, clavulanate has only a small antibiotic effect compared to the antibiotic effect of amoxicillin. But, the clavulanate greatly increases the effect of the amoxicillin by inhibiting an enzyme that degrades the amoxicillin. Without the clavulanate the “time above MIC” for amoxicillin, which correlates with antibiotic effect, is decreased due to enzymatic degradation of the amoxicillin. Thus it is desirable for clavulanate to be present in the body at the same time as the amoxicillin. And, based on understanding that some time is needed for the clavulanate to interact with the enzyme, it is suggested that the most preferred case is when some clavulanate is present at least a short time of 15 minutes or even more before the amoxicillin molecules are present to have an even greater effect.

Both amoxicillin and clavulanate are known to produce adverse gastrointestinal disorders. It is thus preferable that both molecules be absorbed as high in the intestinal tract as possible in order to minimize drug travel distance in the intestines and thereby minimize or prevent drug molecules from exerting their undesirable effects. And, the absorption window for these drugs is in the upper small intestine. Thus, as discussed elsewhere herein, the novel, leaky enteric compositions are ideally suited for delivery of these drugs.

Drug combinations can be separately prepared as individual leaky enteric compositions with individual release rates in gastric fluids, and then combined in any desired ratios. One drug may be released more quickly than the other and therefore be present at a desired site, interacting with an enzyme for one example, before the other drug arrives. Some or none of each drug may be available as IR drug. By way of further illustration, clavulanate is now prepared as any desired salt or as the free molecule in a leaky, enteric-coated bead or other particulate formulation with a controlled dissolution of 80% over 5 hours in gastric fluid. Some IR clavulanate may also be present as part of the 80% or in addition to the 80% to “jump start” drug absorption if desired. Amoxicillin is now prepared separately from the clavulanate as any desired salt or as the free molecule in a different, multiparticulate, leaky enteric-coated bead formulation with a slower overall controlled dissolution of 80% over 7 hours in gastric fluid. Some IR amoxicillin also may be present as part of the 70% or in addition to the 70% to “jump start” drug absorption if desired. In this case there is no additional IR form of drug present. The separately prepared amoxicillin and clavulanate beads are combined in the desired ratio and placed in a gelatin or other capsule and administered to a subject in need of such treatment. Release of the drugs is in a programmed fashion while the beads are in the stomach in gastric fluid and then release of any remaining drug(s) is rapid once the beads are transported into the intestinal fluid, thereby insuring that drug is not entrapped in the composition and unavailable when passing the absorption window. At the same time this embodiment prolonges drug input and drug concentrations in the body compared to immediate-release drug formulations, and demonstrates more rapid absorption of clavulanate than amoxicillin. It is readily understood from the disclosure that this is only one example of combinations of drugs, formulations possible, drug release patterns, and flexibility available to one of ordinary skill in the art that makes it readily possible to provide any ratio of drug combinations and release rates in gastric fluid over any desirable times for any different drug combinations, as desired. And, of course it is clear that drug combinations also can be formulated together in a single, multiparticulate, such as a bead or granule when desired. Preparation of separate bead or granule compositions is not required but is presented as an example of the flexibility of the invention.

Disclosed embodiments of the present invention have been described with reference to particular features of working or prophetic embodiments. The scope of the invention is not limited to these particular features.

1-582. (canceled)

583. A pharmaceutical composition comprising at least one active ingredient in a core and an enteric coating on and/or in the core, the enteric coating further comprising a gastric fluid channel, a gastric fluid channeling agent, or both.

584. The composition according to claim 583 where the enteric coating comprises a gastric fluid channeling agent.

585. The composition according to claim 583 where at least 10% by mass of the active ingredient is released in gastric fluid.

586. The composition according to claim 583 where the active ingredient has a window of adsorption.

587. The composition according to claim 583 where the active ingredient is selected from therapeutic nucleic acids or amino acid sequences, nucleic acids or amino acid derivatives, peptidomimetic drugs, antibiotics, therapeutic ions, vitamins, bronchodilators, anti-gout agents, anti-hypertensive agents, diuretic agents, anti-hyperlipidemic agents or ACE inhibitors, drugs intended for local treatment of the gastrointestinal tract, including anti-tumor agents, histamine (H2) blockers, bismuth salts, synthetic prostaglandins or antibiotic agents, drugs that degrade in the colon, for example, metoprolol, formulations useful for treating gastrointestinal associated disorders selected from peptic ulcer, nonulcer dyspepsia, Zollinger-Ellison syndrome, gastritis, duodenitis and the associated ulcerative lesions, stomach or duodenum neoplasms, prazosin, ketanserin, guanabenz acetate, captopril, captopril hydrochloride, enalapril, enalapril maleate, lysinopril, hydralazide, methyldopa, methyldopa hydrochloride, levodopa, carbidopa, benserazide, amlodipine, nitrendipine, nifedipine, nicardipine, verapamil, acyclovir, inosine, pranobex, tribavirine, vidarabine, zidovudine, AZT, active ingredients that exert a medicinal action at the gastric level, including aluminum hydroxide, magnesium carbonate, magnesium oxide, sucralphate, sodium carbenoxolone, pirenzepin, loperamide, cimetidine, ranitidine, famotidine, misoprostol, omeprazol, or combinations thereof

588. The composition according to claim 583 providing an active ingredient release profile where at least 10% active ingredient by mass is released in gastric fluid, followed by at least 75% release of remaining active ingredient in one hour or less upon contacting intestinal fluid.

589. The composition according to claim 583 where the active ingredient has a window of absorption, and where at least 10% active ingredient by mass is released in gastric fluid, followed by at least 75% release of remaining active ingredient in 30 minutes or less upon contacting intestinal fluid.

590. The composition according to claim 583 where active ingredient release upon contacting gastric fluid is zero order, mixed order or first order, followed by substantially immediate release when remaining composition contacts intestinal fluid.

591. The composition according to claim 584 where the gastric fluid channeling agent is hydrophilic.

592. The composition according to claim 584 where the gastric fluid channeling agent is a sugar.

593. The composition according to claim 584 where the gastric fluid channeling agent is hydrophobic.

594. The composition of claim 583 where the hydrophobic material is selected from talc, magnesium salts, silicon dioxide, hydrocarbons, or combinations thereof.

595. The composition according to claim 583 where the enteric coating has a thickness of 25 μm or less.

596. The composition according to claim 583 where the enteric coating has a thickness of 20 μm or less.

597. The composition according to claim 583 comprising a solid composition.

598. The composition according to claim 583 formulated for oral administration.

599. The composition according to claim 583 further comprising a second formulation designed to provide an active ingredient release profile different from the pharmaceutical composition.

600. The composition according to claim 599 where the second formulation provides immediate release in gastric fluid.

601. The composition according to claim 599 where the active ingredient is amoxicillin or a biologically active salt thereof.

602. The composition according to claim 601 where the active ingredient of the second formulation is clavulanate or a biologically active salt thereof.

603. The composition according to claim 599 where the two formulations are placed in a single capsule or tablet for co-administration.

604. The composition according to claim 583 further comprising an admixture or an overcoat of an immediate release dosage form.

605. The composition according to claim 583 comprising an active ingredient selected from AIDS adjunct agents, alcohol abuse preparations, Alzheimer's disease management agents, amyotrophic lateral sclerosis active ingredient agents, analgesics, anesthetics, antacids, antiarythmics, antibiotics, anticonvulsants, antidepressants, antidiabetic agents, antiemetics, antidotes, antifibrosis active ingredient agents, antifungals, antihistamines, antihypertensives, anti-infective agents, antimicrobials, antineoplastics, antipsychotics, antiparkinsonian agents, antiheumatic agents, appetite stimulants, appetite suppressants, biological response modifiers, biologicals, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfunction active ingredients, fatty acids, gastrointestinal agents, Gaucher's disease management agents, gout preparations, homeopathic remedys, hormones, hypercalcemia management agents, hyponotics, hypocalcemia management agents, immunomodulators, immunosuppressives, ion exchange resins, levocamitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychoactive ingredient agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, or mixtures thereof.

606. The composition according to claim 583 where the active ingredient is selected from abacavir sulfate, abacavir sulfate/lamivudine/zidovudine, acetazolamide, acyclovir, albendazole, albuterol, aldactone, allopurinol, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride, carvedilol, caspofungin acetate, cefazolin, ceftazidime, cefuroxime , chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCl, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

607. The composition according to claim 583 further coated by gelatin or placed inside a gelatin capsule or a tablet.

608. The composition according to claim 583 which increases active ingredient bioavailability at least 20% relative to an immediate release control or a sustained-release control that does not include enteric material.

609. The composition according to claim 583 providing substantially equivalent bioavailability but a reduced active ingredient excretion rate relative to an immediate release control formulation.

610. The composition according to claim 583 providing prolonged drug concentrations for an active ingredient or active ingredients having an absorption window relative to an immediate release or a sustained release control.

611. The composition according to claim 583 where the enteric material is selected from cellulose acetate phthalate (CAP), hydroxypropyl methylcellulose phthalate (HPMCP), polyvinyl acetate phthalate (PVAP), hydroxypropylmethyl cellulose, hydroxypropyl methylcellulose acetate succinate (HPMCAS), cellulose acetate trimellitate, hydroxypropyl methylcellulose succinate, carboxymethyl cellulose, carboxymethyl ethyl cellulose, cellulose acetate phthalate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, polyvinyl acetate phthalate, zein, shellac, copal collophirium, Eduragit L30D55, Eudragit FS30D, Eudragit L100, Eudragit S100, Kollicoat EMM30D, Estacryl 30D, Coateric, Aquateric, or combinations of such materials.

612. The composition according to claim 583 providing controlled in vitro gastric release, followed by pulsatile in vitro intestinal release.

613. The composition according to claim 583 comprising plural active ingredients.

614. The pharmaceutical composition according to claim 583 consisting essentially of a core comprising the at least one active ingredient and the enteric coating comprising a gastric fluid channel.

615. The pharmaceutical composition according to claim 583 consisting essentially of a core comprising the at least one active ingredient and the enteric coating comprising the gastric fluid channeling agent.

616. The pharmaceutical composition according to claim 583 that provides programmed release of the active ingredient, excluding amoxicillin and bisacodyl, the active agent being substantially homogeneously admixed with the at least one enteric material.

617. The pharmaceutical composition according to claim 616 comprising a gastric fluid channeling agent in a weight ratio of from greater than zero percent to about 400% of the weight of the enteric material.

618. The pharmaceutical composition according to claim 583 comprising a sugarbead core.

619. A pharmaceutical composition comprising at least one active ingredient, excluding riboflavin and bisacodyl, and a leaky enteric coating.

620. The pharmaceutical composition according to claim 619 that releases at least 10 percent of active ingredient mass upon contacting gastric fluid, the remaining active ingredient being released substantially completely after contacting intestinal fluid.

621. A method for treating a subject having a condition treatable by an active ingredient, comprising:
providing a pharmaceutical composition comprising an active ingredient suitable for treating the condition and a leaky enteric material; and
treating the subject by administering the pharmaceutical composition to the subject.

622. The method according to claim 621 where the enteric material is provided as a coating having a layer thickness of 25 microns or less.

623. A method for treating a subject having a condition treatable by an active ingredient, comprising:
providing a pharmaceutical composition that provides programmed active ingredient release, the composition comprising at least one active ingredient substantially homogeneously admixed with at least one enteric material comprising a gastric channel, a gastric channeling agent, or both; and
treating the subject by administering the composition to the subject.

624. The method according to claim 623 where the pharmaceutical composition provides programmed active ingredient release by delivering at least a portion of the active ingredient upon contacting gastric fluid followed by substantially complete release of remaining active ingredient thereafter upon contacting intestinal fluid.

625. The method according to claim 624 where the composition releases at least 10 percent of active ingredient mass while contacting gastric fluid, the remaining active ingredient being released substantially completely after contacting intestinal fluid.

626. The method according to claim 623 where the active ingredient is other than riboflavin.

627. The method according to claim 623 where the composition consists essentially of a core comprising the active ingredient suitable for treating the condition, and the leaky enteric coating.

628. The method according to claim 623 where the pharmaceutical composition comprises a sugarbead core.

629. A method for making a gastrically leaky, enteri-coated pharmaceutical composition, comprising:
providing a core comprising an active ingredient;
applying an enteric coating material to the core; and
making the enteric coating leaky.

630. The method according to claim 629 where the enteric coating comprises a gastric fluid channel.

631. The method according to claim 629 where the enteric coating comprises a gastric fluid channeling agent.
